(19) United States
(12) Patent Application Publication
(10) Pub. No.: US 2018/0236339 A1
(43) Pub. Date: Aug. 23, 2018

(54) Head Movement Detection Method and System for Training In Sports Requiring a Swing

(71) Applicant: Tour Pro Tech, LLC, Weston, MA (US)

(72) Inventors: Richard Kosowsky, Weston, MA (US); Timothy Tierney, Weston, MA (US); Michael Kosowsky, Weston, MA (US)

(21) Appl. No.: 15/960,798

(22) Filed: Apr. 24, 2018

Related U.S. Application Data
(63) Continuation of application No. PCT/US2016/061545, filed on Nov. 11, 2016.
(60) Provisional application No. 62/408,268, filed on Oct. 14, 2016.
(60) Provisional application No. 62/253,918, filed on Nov. 11, 2015.

(51) Int. Cl. A63B 69/36; A61B 5/11; A61B 5/00; A63B 24/00; G01S 5/30

(52) U.S. Cl. CPC A63B 69/3614; A61B 5/1123; A61B 5/1126; A61B 5/746; A63B 24/0006; A63B 2220/802; A61B 5/6803; A63B 69/3608; G01S 5/30; A63B 2024/0009; A63B 2024/0012; A61B 5/1114

(57) ABSTRACT

A body part movement detection system and method, for use in training for a sport in carrying out a swing in a target direction, includes a transmitter configured to transmit signals over the course of the swing, using a form of radiation consisting of acoustic and electromagnetic, a first detector configured to receive the signals from the transmitter and to produce a first output related to a first distance of the body part to a first reference location, a controller configured to receive successive first outputs from the detector and to evaluate the successive first outputs in relation to at least one previous first output, to determine whether the body part has experienced motion having a component in the target direction by an amount in excess of a threshold, and an alert system configured to generate an alert to the user when the body part has experienced such motion.

### CROSS-REFERENCE TO RELATED APPLICATIONS

This application is a continuation of International Application No. PCT/US2016/061545 filed on Nov. 11, 2016, which claims the benefit of U.S. Provisional Patent Application No. 62/408,268 filed Oct. 14, 2016 and U.S. Provisional Patent Application No. 62/253,918 filed Nov. 11, 2015, the disclosures of which are incorporated by reference herein in their entirety.

### TECHNICAL FIELD

The present invention relates to motion detection, and more particularly to motion detection for tracking motion of a part of the body in a sport requiring a swing.

### BACKGROUND ART

To develop a good golf swing, a golfer should restrict motion of the head until after the ball is struck. Unfortunately, it is difficult to detect these types of movement in a simple, accurate, non-invasive and real-time manner and without having to train the system.

Some prior art systems involve a sheet of opaque material supported above a golf ball and having a slot therein through which the golfer views a golf ball while swinging, or a hollow tee through which light shines. These systems require the golfer to adjust his/her setup in order to detect head movement forward of a starting position, obscure the view of the ball during the swing, and, by requiring the golfer to re-observe the golfer's prior action, provide information as to head position only after the swing.

Another prior art system for determining an amount and direction of a golfer's head movement during a swing consists of at least two light baffles. The baffles reflect light into the golfer's eyes when the golfer's head is outside of a preset range. The system requires precise setup and the golfer's visual observation in order to determine head movement. And again, the system does not indicate movement without the golfer looking at the system, so the system cannot monitor the head position throughout the entire swing.

Another prior art system involves head gear worn by a golfer that includes motion/position sensing devices from which data is processed to determine a signal indicative of the golfer's head motion. In this system, the motion is detected by the system in the head gear and therefore is difficult to provide head movement information relative to the ball. In addition, the system requires the golfer to train the system with desired and undesirable motions, then compare the stored motions with the current motion.

### SUMMARY OF THE EMBODIMENTS

In accordance with one embodiment of the invention, a method for determining when, in the course of a swing by a user, a body part of the user has experienced motion having a component in a target direction, for use in training for a sport in carrying out the swing at a ball in the target direction, includes repeatedly measuring over the course of the swing, using a form of radiation selected from the group consisting of acoustic and electromagnetic, a first quantity related to a first distance of the body part to a first reference location and a second quantity related to a second distance of the body part to a second reference location. The first and second quantities constituting a measurement set. The method further includes, with respect to each measurement set, calculating a value that is a function of the first and second quantities. The value is indicative of body part position. The method further includes repeatedly evaluating successive values associated with each measurement set in relation to a fiducial value to determine whether the body part of the user has experienced the motion having the component in the target direction by an amount in excess of a threshold. The fiducial value is selected from the group consisting of a default value, at least one previous value, an average of the values, and combinations thereof. The method further includes generating an alert to the user in the event that the body part is determined to have experienced such motion.

In accordance with another embodiment of the invention, a method for determining when, in the course of the swing by a user, a body part of the user has experienced motion having a component in a target direction, for use in training for a sport in carrying out the swing at a ball in the target direction, includes repeatedly measuring over the course of the swing, using a form of radiation selected from the group consisting of acoustic and electromagnetic, a value related to a distance of the body part to a reference location. The value is indicative of body part position. The method further includes repeatedly evaluating successive values associated with each measurement in relation to a fiducial value to determine whether the body part has experienced the motion having the component in the target direction by an amount in excess of a threshold. The fiducial value is a default value, at least one previous value, and/or an average of the values. The method further includes generating an alert to the user in the event that the body part is determined to have experienced such motion.

In accordance with another embodiment of the invention, a body part movement detection system, for use in training for a sport in carrying out a swing at a ball in a target direction, includes a transmitter configured to be positioned on a body part of a user and configured to transmit signals over the course of the swing, using a form of radiation selected from the group consisting of acoustic and electromagnetic. The system further includes a first detector, configured to be placed in a first reference location, configured to receive the signals from the transmitter and to produce a first output related to a first distance of the body part to the first reference location. The first output is indicative of body part position. The system further includes a controller, in communication with the first detector, configured to receive successive first outputs from the first detector and configured to evaluate the successive first outputs in relation to a fiducial value to determine whether the body part has experienced motion having a component in the target direction by an amount in excess of a threshold. The fiducial value is selected from the group consisting of a default value, at least one previous value, and/or an average of the values. The system further includes an alert system configured to generate an alert to the user when the body part has experienced such motion.

In accordance with another embodiment of the invention, a body part movement detection system, for use in training for a sport in carrying out a swing at a ball in a target direction, includes a transmitter configured to be positioned on a body part of a user and configured to transmit signals over the course of the swing, using a form of radiation selected from the group consisting of acoustic and electromagnetic. The system further includes a first detector configured to be placed in a first reference location and configured to receive the signals from the transmitter and to produce a first output related to a first distance of the body part to the first reference location. The system further includes a second detector configured to be placed in a second reference location and configured to receive the signals from the transmitter and to produce a second output related to a second distance of the body part to the second reference location. The first and second outputs constitute a measurement set at a given time. The system further includes a controller, in communication with the first and second detectors, configured to receive the measurement set and to calculate a value that is a function of the first and second outputs, the value being indicative of body part position, and configured to evaluate successive values associated with each measurement set in relation to a fiducial value to determine whether the body part of the user has experienced the motion having the component in the target direction by an amount in excess of a threshold. The fiducial value is selected from the group consisting of a default value, at least one previous value, an average of the values, and combinations thereof. The system further includes an alert system configured to generate an alert to the user when the body part has experienced such motion.

In related embodiments, the method further includes establishing a reference body part position as defining an initial position of the body part at a beginning of the swing, and using the reference body part position to determine the threshold. The body part position may be head position. The method further includes detecting contact with the ball, and ending the generating of the alert when contact is detected. Detecting contact with the ball may include detecting a sound made by the contact. When the form of radiation is acoustic, the acoustic radiation may be ultrasonic radiation. The component in the target direction may include a forward component in a forward target direction and a reverse component in a reverse target direction, and the threshold may include a first threshold and a second threshold. Repeatedly evaluating successive values associated with each measurement set in relation to the fiducial value determines whether the forward component in the forward target direction exceeds the first threshold or the reverse component in the reverse target direction exceeds the second threshold. The body part may be a head.

In related embodiments, the system further includes a sound detection system configured to detect contact with the ball, and the alert system is deactivated when contact is detected. The controller may include a microprocessor. Alternatively, or in addition, the controller includes an analog controller having a comparator. The form of the radiation may be acoustic, and the acoustic radiation may be ultrasonic radiation. The controller may include an adaptive threshold element configured to establish a fiducial body part position as defining an initial position of the body part at a beginning of the swing in order to determine the threshold. The body part may be a head and the transmitter may be configured to be positioned on the head of the user.

In a further related embodiment, the controller is configured to establish the fiducial value corresponding to an initial position of the body part at a beginning of the swing in an adaptive manner depending on actual measurement of position of the body part at the beginning of the swing. Optionally, the fiducial value is established as an average or other combination of body part position measurements at the beginning of the swing.

In another related embodiment, the controller is configured to utilize the threshold that is manually specified. Optionally, the controller is configured to utilize the threshold that is manually specified as pertaining to a selected one of a plurality of skill levels of the user. Alternatively or in addition, the controller is configured to utilize the threshold that is manually specified based on swing type by the user, wherein the swing type is selected from the group consisting of swing, pitch, and putt. Alternatively or in addition, the controller is configured to utilize the threshold that is manually specified as pertaining to a selection based on a plurality of types of swings by the user or user skill levels, wherein the selection is selected from the group consisting of swing, pro swing, pitch, and putt.

In another related embodiment, the transmitter is an ultrasonic transmitter and the system further includes an RF transmitter configured to be positioned on the body part of a user and an RF receiver, coupled to the controller, wherein the controller is configured to receive signals from the RF transmitter that allow the controller and the ultrasonic transmitter to synchronize. The ultrasonic transmitter and the RF transmitter may be configured to produce synchronized ultrasonic and RF bursts respectively, wherein the controller is configured to calculate a first difference, between arrival time at the first detector of a given ultrasonic burst from the ultrasonic transmitter and arrival time at the RF receiver of a corresponding RF burst from the RF transmitter, and a second difference, between arrival time at the second detector of the given ultrasonic burst and arrival time at the RF receiver of the corresponding RF burst, the first and second differences being measures of the first and second distances respectively.

Optionally, the system further includes a housing in which the controller is disposed, the housing having an assembly that supports the first and second detectors, the assembly having a collapsed position in which the first and second detectors are spaced apart by a first distance and an open position in which (i) the system is operative and (ii) the first and second detectors are spaced apart by a second distance that is greater than the first distance. As further option, the system includes a switch configured, when the assembly is moved between the collapsed position and the open position, to toggle the system between an off mode and an on mode. Optionally, the assembly includes first and second shells mounted in slidable relation to one another, the first detector being mounted to the first shell and the second detector being mounted to the second shell.

In another related embodiment, the transmitter is an ultrasonic transmitter and the ultrasonic transmitter is directional and the controller is further configured to defer establishing the fiducial value until after signals from any detector are absent for a specified time interval, wherein looking away from the ball has the effect of preventing an ultrasonic signal from the ultrasonic transmitter from reaching one of the detectors, so that the user must look away from the ball and then return to looking at the ball before the system will establish the fiducial value and begin monitoring the swing.

In yet another related embodiment, the controller is configured to provide hysteresis in resetting of the alert, so that when the alert system has generated the alert as a result of body part movement beyond the threshold, the alert system is configured not to generate a subsequent alert unless the body part has moved inside a narrower threshold and remained there for a predetermined amount of time.

In another related embodiment, the system includes a transceiver configured to couple a portable computing device to the controller, wherein, when the computing device is executing an application configured to communicate with the controller, the application can store and display data relating to performance of the user with respect to the system. The transceiver may, for example, use the Bluetooth protocol. Optionally, the application is further configured to provide a user interface for configuration of parameters relating to operation of the system. Also optionally, the application is further configured to communicate with a server system that stores the data relating to performance of the user with respect to the system.

In another related embodiment, the controller is further configured to evaluate successive values associated with each measurement set in relation to the fiducial value to determine whether the body part of the user has experienced motion having a component in a direction perpendicular to the target direction by an amount in excess of a second threshold. The body part of the user may be a head and the transmitter may be configured to be positioned on the head of the user.

### BRIEF DESCRIPTION OF THE DRAWINGS

The foregoing features of embodiments will be more readily understood by reference to the following detailed description, taken with reference to the accompanying drawings, in which:

FIG. 1 schematically shows a top view of a head movement detection system having one detector in two alternate locations on an axis parallel to the target direction according to embodiments of the present invention.

FIGS. 2A and 2B schematically show a top view and a side view, respectively, of a head movement detection system having two detectors on an axis parallel to the target direction according to embodiments of the present invention.

FIG. 3 schematically shows a top view of a head movement detection system having two detectors that communicate information to a control unit according to embodiments of the present invention.

FIG. 4A schematically shows a top view of a head movement detection system where detectors and control unit are attached to a golf bag stand according to embodiments of the present invention.

FIG. 4B schematically shows a side view of a head movement detection system where detectors and control unit are attached to a golf cart according to embodiments of the present invention.

FIG. 5 shows a schematic block diagram of a head movement detection system with an ultrasonic transmitter according to embodiments of the present invention.

FIG. 6 shows a schematic block diagram of an ultrasonic detector according to embodiments of the present invention.

FIG. 7 schematically shows a control unit, for use in a head movement detection system, having a sound detection system according to embodiments of the present invention.

FIG. 8 shows a schematic block diagram of the sound detection system shown in FIG. 7 according to embodiments of the present invention.

FIG. 9 schematically shows two types of controllers that may be used in a control unit of a head movement detection system according to embodiments of the present invention.

FIG. 10 shows a schematic block diagram for a method of determining when the head of a user has experienced motion using a two detector system according to embodiments of the present invention.

FIG. 11 shows a schematic block diagram for a method of determining when the head of a user has experienced motion using a one detector system according to embodiments of the present invention.

FIG. 12 schematically shows a top view of a head movement detection system with a control unit positioned on the head of a user according to embodiments of the present invention.

FIGS. 13A and 13B schematically show a perspective front view and back view, respectively, of a control unit that may be opened and closed, in the closed or off state, for use in a head movement detection system according to embodiments of the present invention.

FIGS. 14A and 14B schematically show a perspective front view and back view, respectively, of a control unit that may be opened and closed, in the open (or on) state, for use in a head movement detection system according to embodiments of the present invention.

FIG. 15 schematically shows an accessory unit for housing an ultrasound transmitter for use in a head movement detection system according to embodiments of the present invention.

FIG. 16 is a flowchart describing an algorithm that may be used to control a control unit, for use in a head movement detection system according to embodiments of the present invention.

FIGS. 17A-17C schematically show a top view of a head movement detection system having two detectors on an axis parallel to the target direction where the user can select a variety of combinations of location thresholds according to embodiments of the present invention.

FIG. 18 is a flowchart describing an algorithm that may be used in the implementation of a variety of combinations of location thresholds according to embodiments of the present invention.

FIG. 19 is a flowchart describing an algorithm that may be used to determine if the user is ready to start a swing according to embodiments of the present invention.

FIG. 20 schematically shows a top view of a head movement detection system having two detectors on an axis parallel to the target direction where the alert system includes an algorithm for preventing subsequent alerts until specified motion has occurred after a first alert according to embodiments of the present invention.

FIG. 21 is a flowchart describing an algorithm that may be used to prevent subsequent alerts until specified motion has occurred after a first alert in a head movement detection system, according to embodiments of the present invention.

FIG. 22 is a flowchart describing an algorithm that may be used to detect contact in a head movement detection system, having a sound detection system, according to embodiments of the present invention.

FIG. 23 schematically shows a top view of a head movement detection system having two detectors and a control unit on an axis parallel to the target direction and a transmitter positioned on the head of a user, using both ultrasonic and radio frequency signals, according to embodiments of the present invention.

FIG. 24 is a flowchart describing an algorithm that may be used to control a transmitter in a head movement detection system according to embodiments of the present invention.

FIG. 25 schematically shows a head movement detection system that communicates with a portable computing device according to embodiments of the present invention.

FIG. 26 schematically shows a head movement detection system that communicates with a network, and, through the network, to computing devices, according to embodiments of the present invention.

### DETAILED DESCRIPTION OF SPECIFIC EMBODIMENTS

Definitions. As used in this description and the accompanying claims, the following terms shall have the meanings indicated, unless the context otherwise requires:

A “reference location” means a position that is static when detecting motion of the head of a user in the course of a swing.

A “fiducial value” is a value indicative of head position at the start of a swing.

An output is provided “repeatedly” over time if the output is continuous over time or if the output is provided in a stream of successive samples over time.

Two outputs occur “at a given time” if they pertain to instants in time that are approximately adjacent.

A head of the user has “experienced motion having a component in the target direction” when the component has an absolute value greater than a threshold designated as insubstantial. Under such a circumstance, motion in the target direction or away from the target direction qualifies as “motion having a component in the target direction.”

A “set” includes at least one member.

A “swing” means the movement of at least one arm of a user for the purpose of making contact with an object. The swing may include a bunt, drive, putt, full swing, chip, stroke, forehand stroke, backhand stroke, serve, lob, strike, punch, volley, etc.

A “practice mode” is an operating mode of the system to be used in training a particular type of swing.

Various embodiments of the present invention determine when the head of a user has experienced motion by an amount in excess of a threshold over the course of a swing. For example, embodiments of the present invention provide a system which detects and alerts a golfer of forward lateral head movement along a path generally parallel to a target line as the golfer executes a golf swing, which solves the problems and overcomes the drawbacks and deficiencies of prior art golf swing assistance systems. The system of various of these embodiments includes two or more radiation sensors placed along a path generally parallel to a target line and in communication with a control unit and a third sensor placed or worn on the head. The sensors interact with one another to determine if and at what point the head of the user moves forward during a swing and alerts the user immediately. In a further embodiment, the system has a feature by which it ignores the movement of the head once contact has been made with the ball or object, a feature that eliminates false alarms, because, after contact with the ball has been made, it does not matter where the head moves. The system of these embodiments uses two detectors to calculate the relative distance of a transmitter. The starting position of the head, deviation from which in the course of a swing creates an alerting condition, may be the midpoint between the two detectors. Alternatively, the system may determine an adaptive starting position of the head, for which the relative distance of the head from the two detectors is initially calculated to derive a starting position, and deviation from the starting position forms the basis for alerting. The position may be calculated by the system and set for each swing at the convenience of the user. In various embodiments, the system provides an initialization mechanism by deferring calculation of the starting position until the user has looked away from the ball for a specified period. In various embodiments, the threshold is user selectable. The threshold arrangement in various embodiments includes hysteresis, so that when the alert system has generated the alert as a result of head movement beyond the threshold, the alert system is configured not to generate a subsequent alert unless the head has moved inside a narrower threshold and remained there for a predetermined amount of time. In a further embodiment, the system is configured to detect motion both parallel to and perpendicular to the target direction. In further embodiments, the system may include connectivity with a smartphone or a computer network. Details of illustrative embodiments are discussed below.

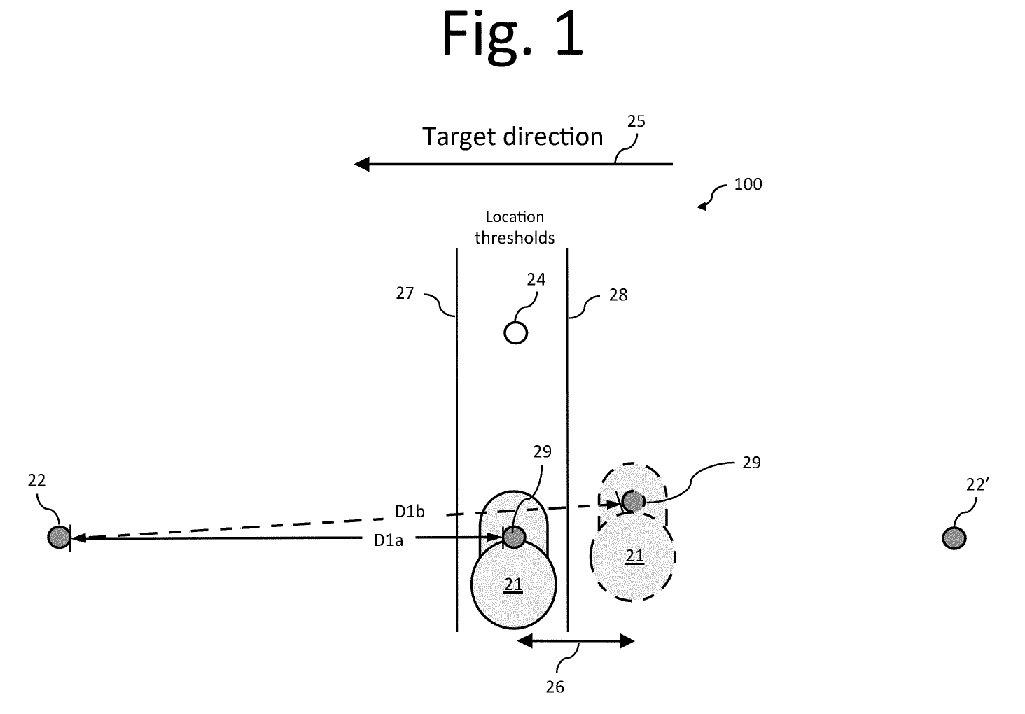

FIG. 1 shows an embodiment of a head movement detection system 100 that determines whether the head 21 of the user has moved outside one or more thresholds 27, 28. The system 100 includes a detector 22 configured to be placed in a first reference location, and a transmitter 29 configured to be positioned on the head 21 of the user. In preferred embodiments, the transmitter 29 is coupled to a head covering, e.g., a hat or helmet. The transmitter 29 transmits signals to the detector 22, e.g., using acoustic and/or electromagnetic radiation, over the course of the swing at the ball 24, and the detector 22 receives the signals from the transmitter 29. The detector 22 then produces an output related to a first distance, D1a, of the head to the first reference location. The detector 22 and transmitter 29 are in a line parallel to a target direction 25, so as to detect motion parallel to the axis of motion 26. As shown in FIG. 1, the detector may be placed in location 22 in the target direction 25 or the detector may be placed in location 22′ opposite to the target direction. The transmitter 29 and detector 22 repeatedly send and receive signals by which can be determined location of the head of the user over the course of the swing. These signals may be sent and received over timed intervals or continuously.

The system 100 also includes a controller (discussed in more detail below), in communication with the detector 22; the controller receives successive outputs from the detector 22 and evaluates the successive outputs in relation to a fiducial value to determine whether the head 21 has experienced motion having a component in the target direction 25 exceeding the one or more thresholds 27, 28. The fiducial value may be at least one previous output, (e.g., the most recently qualified output, where an output only qualifies if it is close to the previous output), a default value (e.g., zero), an average of the outputs, or a desired combination of these items (e.g., an average of the set of five most recent qualified outputs). In accordance with a further related embodiment, the system 100 has a sync signal for timing. The sync signal may be provided, e.g., by a physical wire or by RF transmission. The component of head motion measured in the target direction 25 may include a forward component in a forward target direction and/or a reverse component in a reverse target direction opposite to the forward target direction. The controller may use a reference head position received from the detector 22 defining an initial position of the head at the beginning of the swing. The controller may use the reference head position to determine the one or more thresholds 27, 28. The threshold 27, 28 defines the maximum amount of movement the head 21 should experience over the course of the swing. The thresholds 27, 28 may be calculated using various values, e.g., the starting position of the head at the beginning of the swing, the height of the user, and the type of swing of the user. In some embodiments, the thresholds 27, 28 may be adjusted based on variables such as the skill of the player, the type of sport, the physical placement of the detector 22, and the intent of the swing. For example, in some embodiments, the calculated thresholds 27, 28 may be larger, e.g., if the user is a beginner instead of an intermediate or advanced user. In other embodiments, the calculated thresholds 27, 28 may be smaller if a golfer is putting as opposed to chipping or driving the ball. In other embodiments, the calculated thresholds 27, 28 may be smaller, e.g., if the user was taller than the average person, such that the thresholds 27, 28 would be calculated to be inversely proportional to the height of the user.

The system 100 also includes an alert system (discussed in more detail below) that generates an alert to the user when the head 21 of the user undergoes motion that is in excess of the one or more thresholds 27, 28. For example, as shown in FIG. 1, if the head 21 is determined to have moved from distance D1a to distance D1b, then the alert system notifies the user that the head has moved by an amount that is in excess of the threshold 28. This alert can be a sensory alert including alerts communicated visually, audibly, or through touch, e.g., lights, sounds, and/or vibrations.

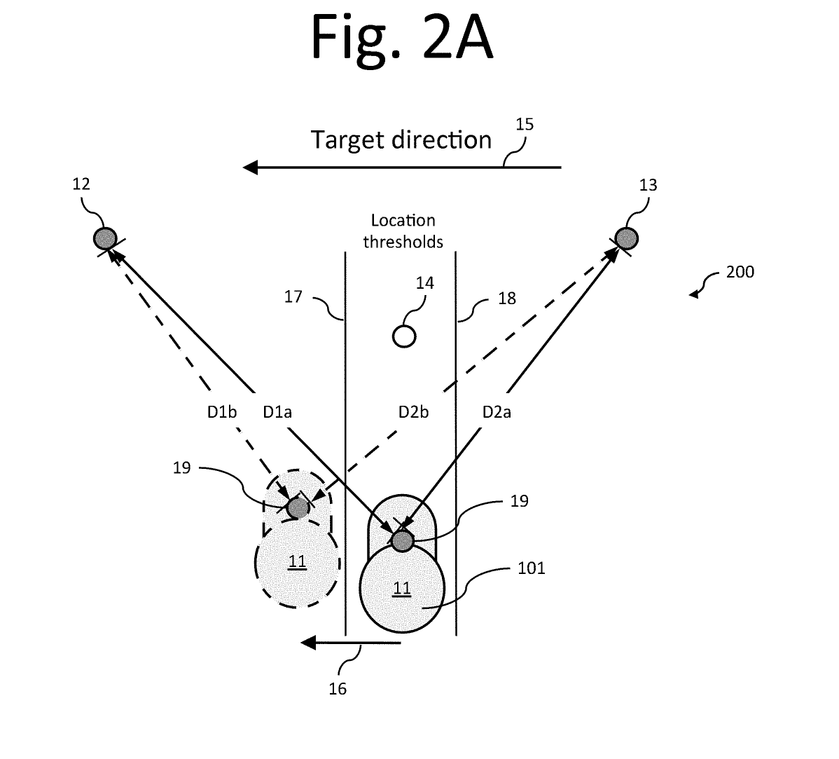

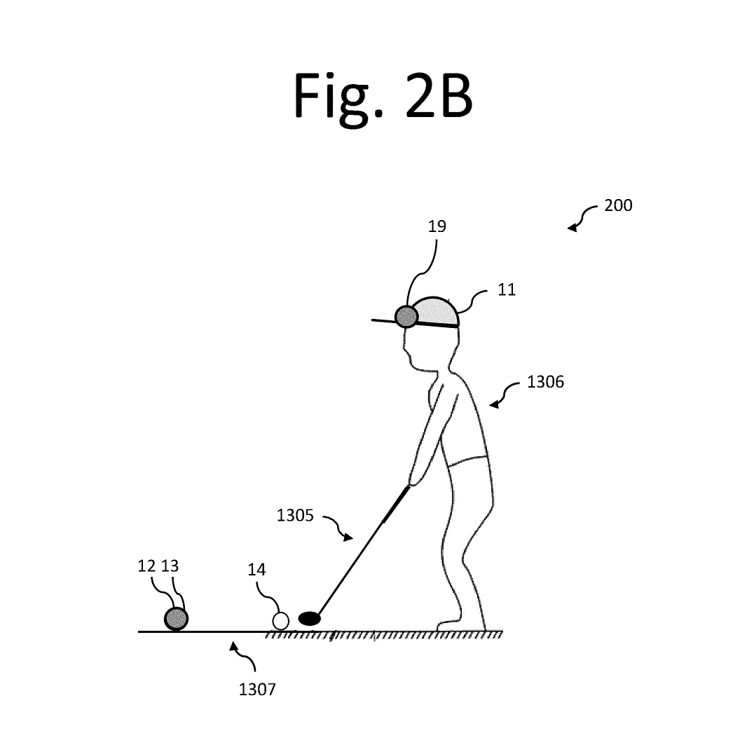

FIGS. 2A and 2B show a top view and side view, respectively, of another embodiment of a head movement detection system 200 where the user 1306 swings a golf club 1305 at a ball 14. The head movement detection system 200 is similar to the head movement detection system 100 discussed above in connection with FIG. 1, except two or more detectors 12, 13 and one transmitter 19 are used. The transmitter 19 transmits signals, e.g., using acoustic and/or electromagnetic radiation, over the course of the swing at the ball 14 to the two or more detectors 12, 13. As shown in FIG. 2A, the detectors 12, 13 are positioned at two separate reference locations on an axis 16 parallel to the target direction 15. In a manner similar to the system 100, the transmitter 19 sends signals to the detectors 12, 13, and the detectors 12, 13 receive the signals from the transmitter 19 in order to determine head movement in the target direction 15 (e.g., in the forward or reverse target direction). For example, when two detectors are used, the first detector 12 is placed in a first reference location and the second detector 13 is placed in a second reference location, such as shown in FIG. 2A. In this example, the first detector 12 produces a first output related to a first distance, D1a, of the head 11, and the second detector 13 produces a second output related to a second distance, D2a, of the head 11. The first and second outputs constitute a measurement set at a given time.

The system 200 also includes a controller (discussed in more detail below), in communication with the detectors 12, 13, which receives the measurement set and calculates a value that is a function of the first and second outputs and that is indicative of head position. The controller evaluates successive values associated with each measurement set in relation to a fiducial value, to determine whether the head 11 has experienced motion having a component in the target direction 15 that exceeds one or more thresholds 17, 18. The fiducial value may be at least one previous output, a default value (e.g., zero), an average of the outputs, or a desired combination of these items. For example, if the head moves during the course of the swing, the first detector 12 produces first outputs related to a first distance, D1b, of the head and the second detector 13 produces second outputs related to a second distance, D2b, of the head. As discussed above, the thresholds 17, 18 may be calculated. The controller may use a myriad of factors, e.g., the user's initial position, the user's height, skill level, dexterity, intent, and type of sport, to calculate thresholds. The thresholds 17, 18 may be determined by the physical placement of the detectors 12, 13. For example, the thresholds 17, 18 may become smaller if the detectors are placed further apart, and the thresholds 17, 18 may become larger if the detectors are placed closer together. Some embodiments allow the user to manually input factors into the controller to ensure accurate calculations. The factors may be adjusted manually by the user or may be automatically adjusted based on perceived increased skill level of the user over time. The controller may use a fiducial head position, received from the detectors 12, 13, defining an initial position of the head at the beginning of the swing. The controller uses the fiducial head position to determine the one or more thresholds 17, 18. Alternatively, the fiducial head position may be indicated manually. Manual indication may include the user's pushing a button to signal to the system 200 that the system 200 should begin detecting and comparing head position. Alternatively, or additionally, manual indication may include the user's moving in a specific way to signal to the system 200 to begin detecting head position. The position may also be calculated passively, with an indicator to alert the user that the device has begun detecting the head position at a starting time. This indicator may be a visual, audio, and/or other sensory indicator, such as a light, sound, and/or vibrational indication.

The system 200 also includes an alert system (discussed in more detail below) that generates an alert to the user when the head 11 of the user moves in excess of the one or more thresholds 17, 18. For example, as shown in FIG. 2A, if the head 11 is determined to have moved from distance D1a, D2a to distance D1b, D2b then the alert system notifies the user that the head has moved in excess of the threshold 17. This alert can be a sensory alert including alerts communicated visually, audibly, and/or through touch, e.g., lights, sounds, and/or vibrations.

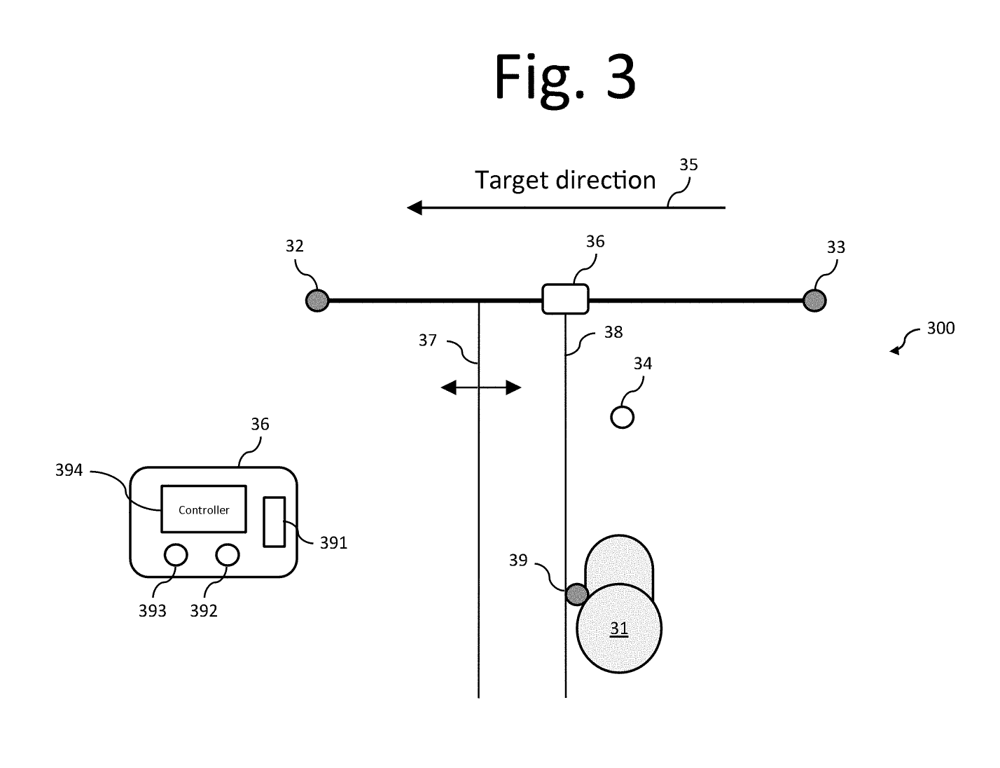

FIG. 3 shows a head detection system 300 having one transmitter 39 and two detectors 32, 33 that communicate information to a control unit 36. The transmitter 39 and detectors 32, 33 of system 300 are similar to those discussed above in systems 100 and 200. The control unit 36 includes a controller 394, such as discussed above with regard to systems 100 and 200. The control unit 36 indicates to the user when the controller 394 has started to receive input about the position of the head 31 over the course of the swing. The control unit 36 may indicate, to the user, that it is ready, to assess head motion in the swing, by means of visual, audio, and/or other sensory alerts, such as a light 393, a buzzer 392, and/or a sound alert. The controller 394 uses the initial transmitter position 38 to calculate the distance the head 31 can move in a target direction 35, setting a threshold 37. If the head 31 of the user exceeds the calculated threshold 37, the control unit 36 produces an output which signals that the user has experienced head motion exceeding a calculated threshold 37. In some embodiments, the control unit 36 may determine that the user has exceeded the threshold when the transmitter 39 moves outside of the calculated threshold 37 during the course of the swing. The control unit alerts the user about this excess movement through audio, visual, and/or sensory signals. These signals include light 393, a buzzer vibration 392, and/or a sound. In some embodiments, control unit 36 may be powered by a battery 391.

Once contact is made with the ball 34, the control unit 36 may stop alerting the user that the head 31 has moved outside of the calculated threshold 37. The controller 394 may determine when the user swings and misses the ball 34 if the time, between the user's starting the swing and the user's ending the swing, exceeds a certain amount of time. Making contact with the ball 34 may also signal the end of a swing. In some embodiments, the control unit 36 may use sound to detect the user's swinging and making contact with the ball 34. The control unit 36 may detect a pre-programmed frequency associated with a user's swinging and making contact with the ball 34 with the proper sporting equipment.

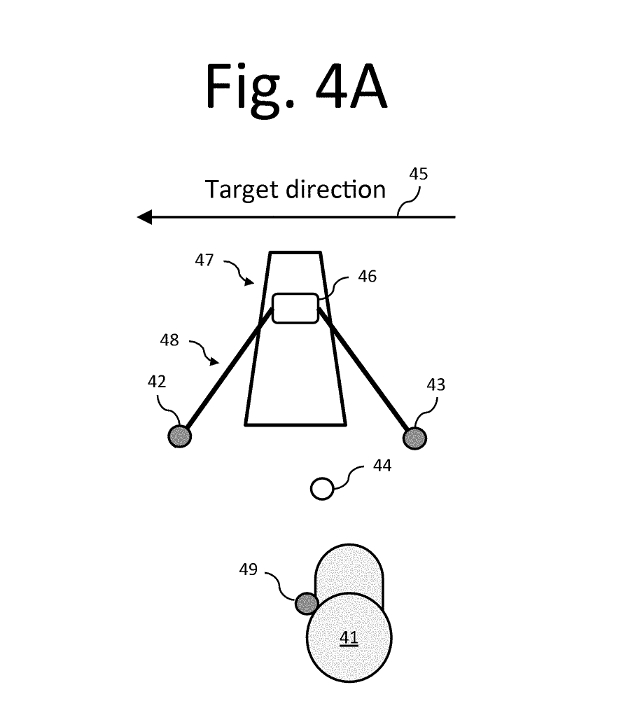

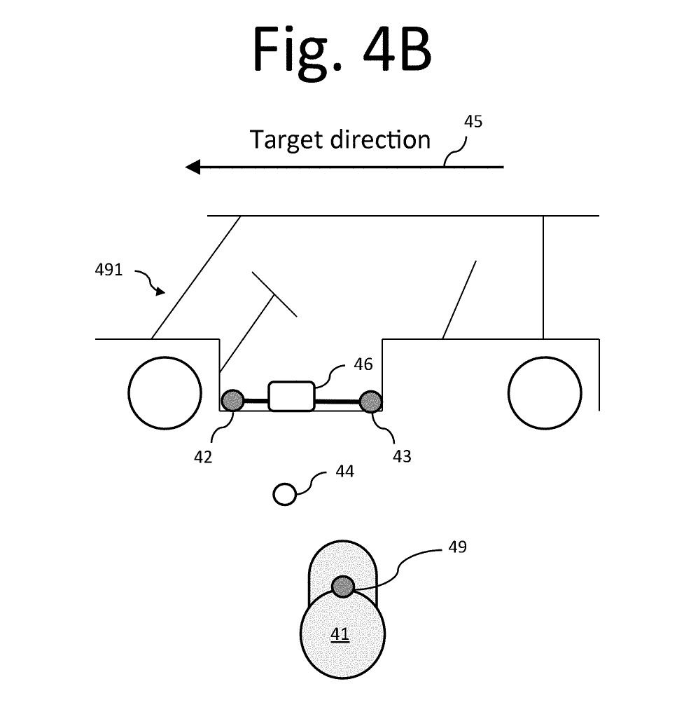

The head movement detection system 100, 200, 300 may be incorporated into various components. The components may be attached to a golf bag, such as shown in FIG. 4A, where the control unit 46 is attached to the golf bag 47 and the one or more detectors 42, 43 may be in communication with the control unit 46 and attached to legs 48 of the golf bag 47. Alternatively, the components may be attached to a golf cart, as shown in FIG. 4B, where the control unit 46 and the one or more detectors 42, 43 attach to the golf cart 491.

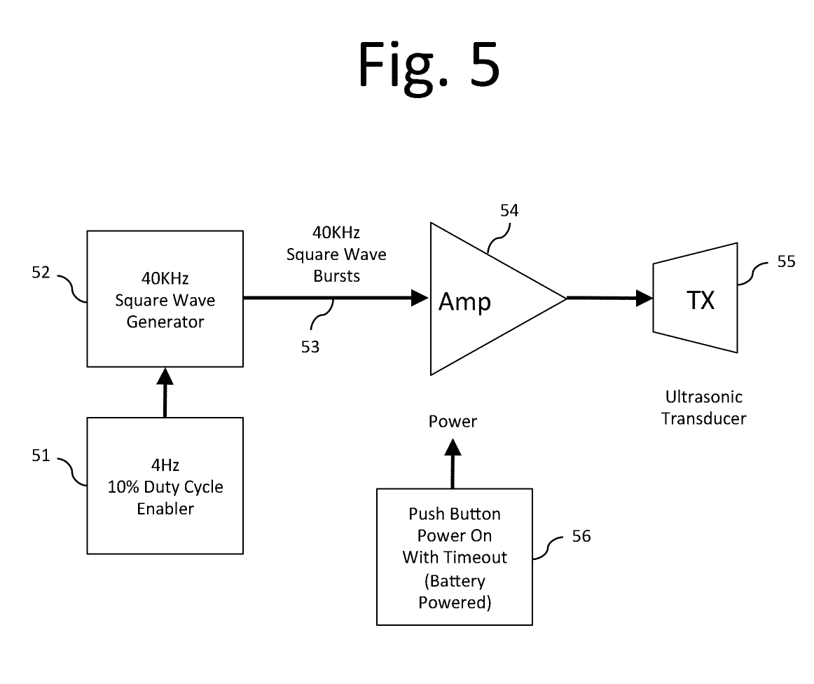

FIGS. 5-9 show specific implementations of various components used in the head movement detection systems 100, 200, 300 discussed above. For example, FIG. 5 shows a transmitter that may use acoustic radiation to transmit, to at least one reference location over the course of the swing, signals related to the distance of the head of the user. Other forms of radiation may also be used, such as electromagnetic radiation. In various embodiments, a transmitter may be an ultrasonic transmitter that includes a square wave generator 52 (e.g., a 40 KHz square wave generator) with an amplifier 54 and an ultrasonic transducer 55 to transmit signals over the course of the swing. The transmitter may emit intermittent signals (e.g., using a duty cycle enabler 51) to transmit the head position of the user to one or more detectors.

Alternatively, the transmitter may use electromagnetic radiation, such as RF radiation, to transmit signals to one or more detectors. For example, rather than transmitting bursts, packets of a given frequency (e.g., 868 MHz frequency) may be used along with an amplifier 54 and an antenna (not shown) to transmit the signals to one or more detectors. The transmitter may emit constant signals or intermittent signals. For example, the transmitter may transmit bursts every 100, 250, or 500 milliseconds. In some embodiments both acoustic and electromagnetic radiation are employed.

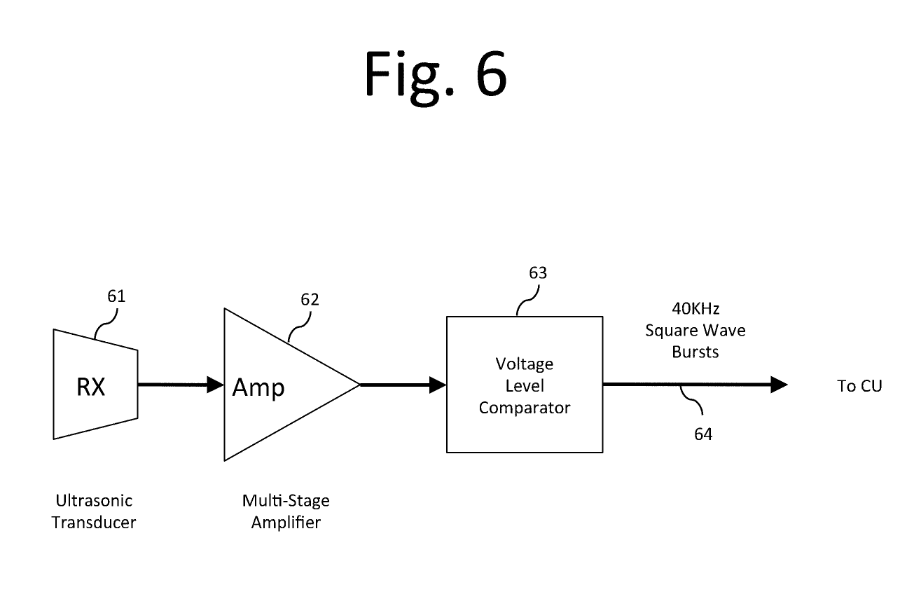

FIG. 6 shows a detector that may be used to receive signals from the transmitter. As discussed above, the transmitter may use acoustic and/or electromagnetic radiation. When using an ultrasonic transmitter, the detector includes an ultrasonic transducer 61, a multi-stage amplifier 62, and a voltage level comparator 63, that communicates an output 64 to a controller in a control unit. For example, the comparator 63 transmits the signals to the controller in 40 KHz square wave bursts 64. Alternatively, when using an RF transmitter, the detector may use an antenna and an amplifier with auto gain control (AGC) to receive the RF transmissions. In this case, the detector outputs to the controller the received signal strength indicators (RSSI) for every packet received. This may be communicated in bursts or continuously.

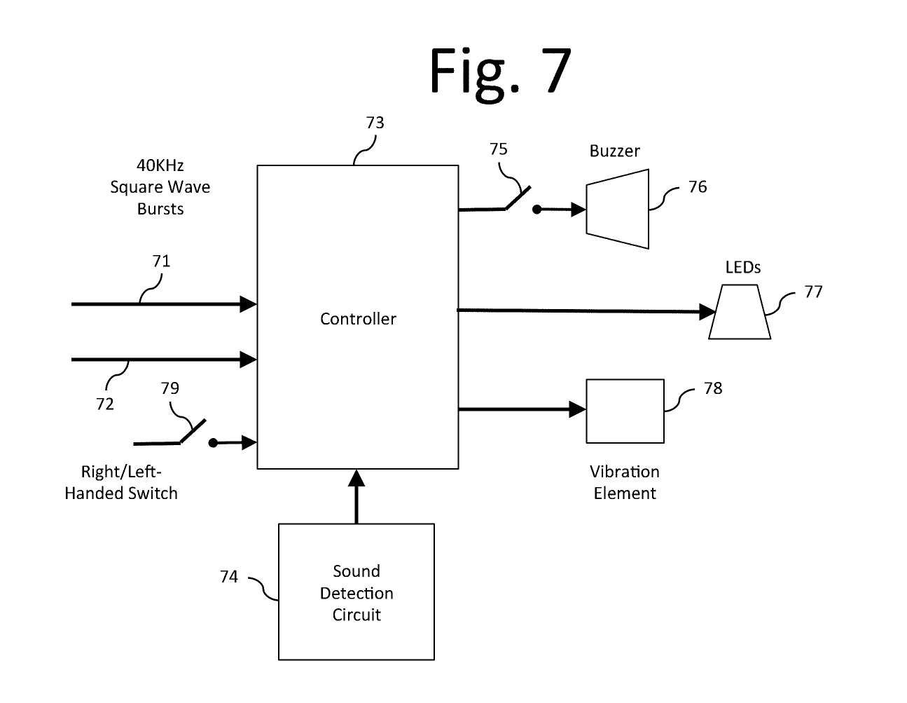

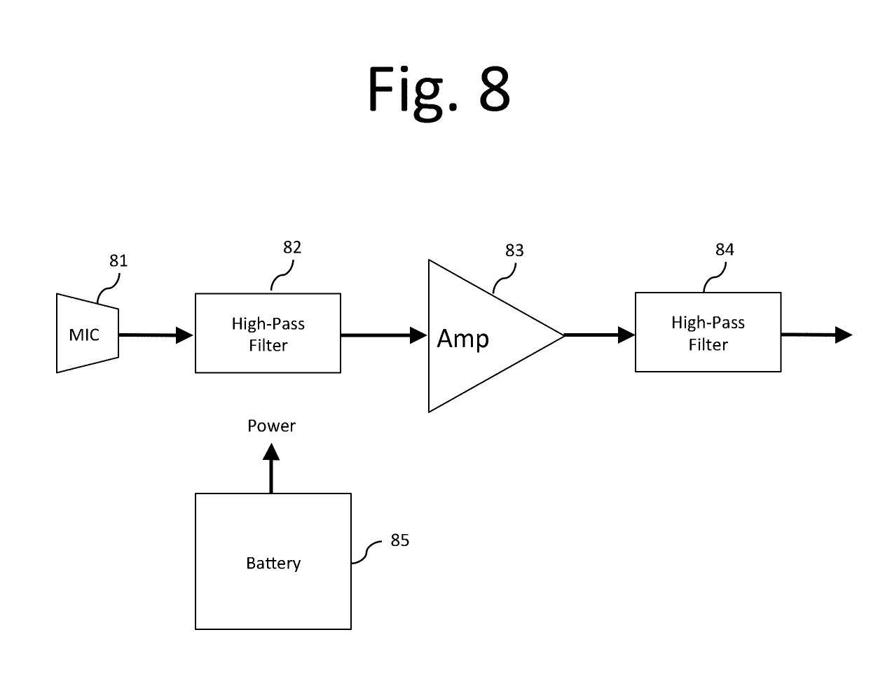

FIG. 7 shows, in accordance with an embodiment of the present invention, a control unit having a controller 73 that receives at least one output 71 from a detector or multiple outputs 71, 72 from multiple detectors. The controller 73 also receives input from a sound detection circuit 74 (shown in more detail in FIG. 8), which detects a sound, indicating when the user makes contact with the ball. The controller 73 receives outputs from one or more detectors indicating the position of the head of the user at a given time. For example, the controller 73 may receive square wave bursts from an ultrasonic transducer. In some embodiments, the one or more detector outputs 71, 72 are received by the controller 73 and the controller 73 may need to calculate a value related to the distance of the head to one or more reference locations using the one or more detector outputs 71, 72. For example, the controller 73 may determine the position of the head by comparing the time the signals are received or by comparing received signal strength indicators (RSSI) from RF signals to determine the position of the head of the user at a given time. If the controller 73 determines the head of the user has exceeded the calculated threshold, the control unit may generate an alert to the user. This alert may be a visual, audio, and/or sensory alert, using a buzzer 76, light 77, and/or vibration element 78. A switch 75 may enable or disable the buzzer 76 from producing sound as part of the alert. The light 77 may be one or more LEDs. The light 77 may indicate to the user that the controller 73 has determined a reference head position of the user at the beginning of the swing. The light 77 may indicate that the head movement detection system 100, 200, 300 is on or operational.

The controller 73 may receive input about whether the user is left-handed or right-handed and may use this input to calculate the thresholds and/or whether the head of the user has exceeded the thresholds over the course of the swing. This input may be programmed into the controller 73 using a button or switch 79, or may be automatically detected by the controller 73. For example, the control unit shown in FIG. 7 may detect a user swinging from the front of the control unit or from the back. A user may need to activate a right-handed/left-handed switch in some embodiments for the control unit to detect a change in the swing. In other embodiments of the invention, the control unit may detect the change automatically and adjust the calculated thresholds accordingly.

FIG. 8 shows a sound detection circuit that may be used to indicate when the user makes contact with the ball. The sound detection circuit includes a microphone 81 to detect a sound indicating contact with the ball. This sound may be programmed based on the type of sport or sporting equipment used for a swing. The sound detection circuit may also be programmed to detect multiple types of contact with the ball for use in multiple sports. The sound detection circuit includes a high-pass filter 82, an amplifier 83, and another high pass filter 84, to filter out background noise and any interference, in order to produce an output received by the controller, which determines whether the user has made contact with the ball during the swing. In some embodiments, once contact is detected, the controller stops alerting the user to the position of the head of the user. In some embodiments, the sound detection circuit may be powered with a battery 85.

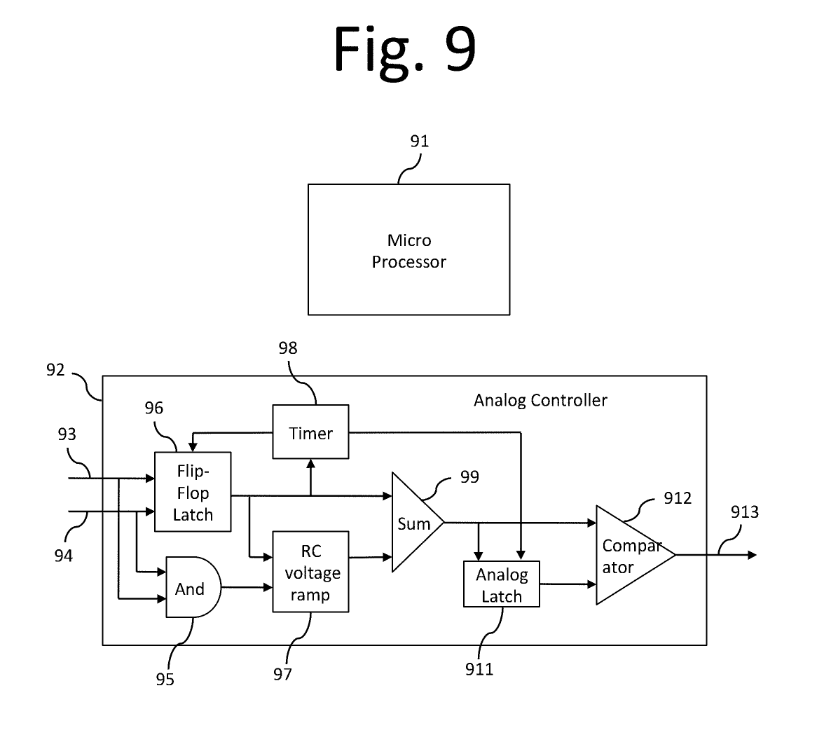

FIG. 9 shows two implementations of a controller that may be used in the head movement detection systems 100, 200, 300 discussed above. The controller may be implemented as a microprocessor 91 or as an analog controller 92. When using an ultrasonic transmitter and one or more detectors, the microprocessor 91 uses a time comparison to determine the position of the head relative to one or more reference locations. In the analog controller 92, the controller 92 receives two inputs 93, 94 from the one or more detectors related to the distance of the head to a reference location using various components, e.g., a flip-flop latch 96, and delay 95, RC voltage ramp 97, timer 98, sum comparator 99, analog latch 911, and comparator 912, and compares the one or more inputs 93, 94 with a fiducial input value to determine whether the head has moved outside of a calculated threshold. The fiducial input value may be a default value (e.g., zero), at least one previous value, an average of the values, or a desired combination of these items. The analog controller 92 then sends the output 913 to the alerting system if the user should be notified that the head movement exceeded the one or more thresholds.

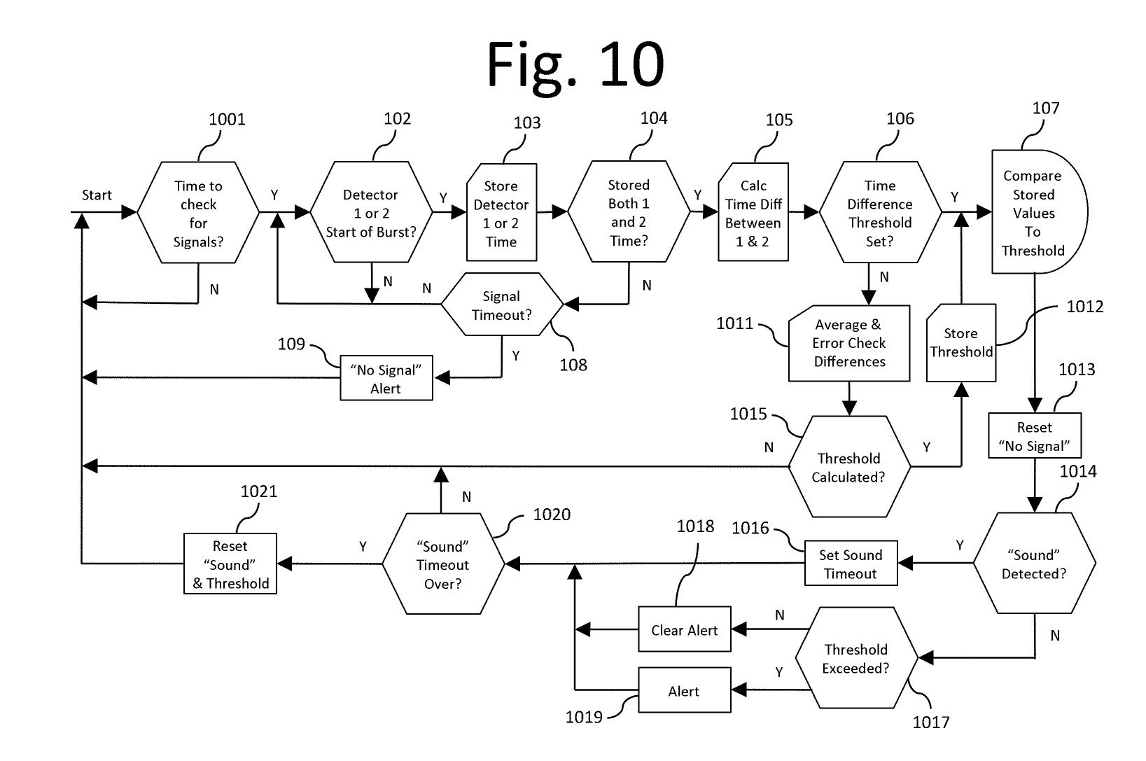

FIG. 10 shows a method of detecting the position of a head that may be used with a head movement detection system having two detectors, such as shown in FIGS. 2A through 4B. The method begins, in process 1001, by determining if it is time to check for signals from one or more detectors. If it is time to check for signals, the method determines if detector 1 or detector 2 has started emitting bursts in process 102. If yes, then the method stores the time that the burst is received by detector 1 or detector 2 in process 103. If the method does not detect that detector 1 or detector 2 has started emitting bursts, then the method repeats process 102. After storing the time related to when the burst is received by one detector in process 103, the method determines if both detector 1 and detector 2 have a time stored. If not, the method repeats processes 102 and 103 until times are stored for both detectors in process 104. If the process takes too long to store both values, then the method times out in process 108 and notifies the user of a “no signal” alert in process 109. In this case, the method may reset and start over again in process 1001.

If the method stores times for both detector 1 and detector 2 in process 104, then the time difference is calculated, using the controller as discussed above, between the stored time for detector 1 and the stored time for detector 2 in process 105. In process 106, the method determines if the time difference threshold has been set. If not, the method runs average and error check differences in process 1011 using the controller and then determines if the threshold can be calculated in process 1015. If the threshold cannot be calculated, for example the time difference computed at process 105 exceeds a system parameter, or too few time differences have been computed since the threshold was last reset, or the time difference computed at process 105 differs substantially from previous time differences computed since the threshold was last reset, then the method returns to process 1001. If the threshold can be calculated, then the threshold value is stored in process 1012, and the method returns to process 106, which determines that the threshold is now set. Once the threshold has been set, the method compares the stored values to the one or more thresholds in process 107 using the controller as discussed above. The method then resets a “no signal” switch in process 1013 and determines if a sound has been detected in process 1014 using the sound detection circuit as discussed above. If a sound is detected in process 1014 indicating that the user has made contact with the ball, a sound timeout is set in process 1016 and the method determines if the sound timeout is over in process 1020. If the sound timeout is over, then the method resets the sound timeout and the one or more thresholds in process 1021 and the method begins again in process 1001. If the sound timeout is not over, then the method begins again in process 1001 and does not reset the sound timeout and thresholds. In process 1014, if a sound is not detected, then the method determines if the one or more thresholds have been exceeded in process 1017. If the head motion of the user has exceeded the one or more thresholds, then an alert is generated by the alert system in process 1019. In process 1017, if the head motion of the user did not exceed the one or more thresholds, then the alert is cleared in process 1018 by the controller. Whether the user is alerted in process 1019 or the alert is cleared in process 1018, the method returns to process 1020 to determine if the sound timeout is over. If the sound timeout is over, then the method resets the sound timeout and the one or more thresholds in process 1021 and the method begins again in process 1001. If the sound timeout is not over, then the method begins again in process 1001 and does not reset the sound timeout and thresholds.

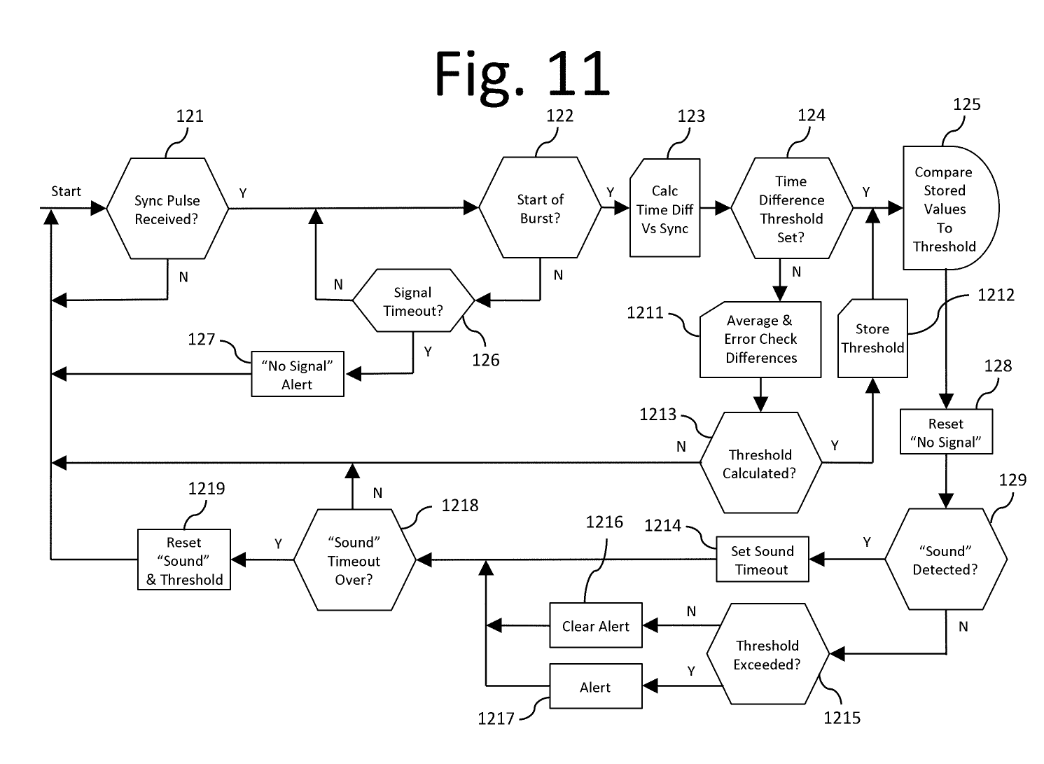

FIG. 11 shows a method of detecting the position of a head that may be used with a head movement detection system having one detector and transmitter, such as shown in FIG. 1. The method of FIG. 11 is substantially the same as the method of FIG. 10 once the process determines in process 124 (in a manner comparable to process 106 in FIG. 10) if the time difference threshold has been set . The method begins by determining if a sync pulse is received in process 121. If the sync pulse has not been received, then the method repeats process 121. If the sync pulse has been received, then the method determines if the detector has started emitting bursts in process 122. If the method does not detect that that detector has started emitting bursts, then the method repeats process 122. If the detector takes too long to start emitting bursts, then the method times out in process 126 and notifies the user of a “no signal” alert in process 127. In this case, the method may start over again in process 121. If the detector starts emitting bursts in process 122, then the method calculates the time difference between the sync pulse and the received burst in process 123 using the controller. As mentioned above, the method then determines in process 124 if the time difference threshold has been set. If not, the method runs average and error check differences in process 1211 using the controller and then determines if the threshold can be calculated in process 1213. If the threshold cannot be calculated, for example the time difference computed at process 123 exceeds a system parameter, or too few time differences have been computed since the threshold was last reset, or the time difference computed at process 123 differs substantially from previous time differences computed since the threshold was last reset, then the method returns to process 121. If the threshold can be calculated, then the threshold value is stored in process 1212, and the method returns to process 124, which determines that the threshold is now set. Once the threshold has been set, the method compares the stored values to the one or more thresholds in process 125, using the controller as discussed above. The method then resets a “no signal” switch in process 128 and determines if a sound has been detected in process 129 using the sound detection circuit as discussed above. If a sound has been detected in process 129, indicating that the user has made contact with the ball, a sound timeout is set in process 1214 and the method determines if the sound timeout is over in process 1218. If the sound timeout is over, then the method resets the sound timeout and the one or more thresholds in process 1219 and the method begins again in process 121. If the sound timeout is not over, then the method begins again in process 121 and does not reset the sound timeout and thresholds. In process 129, if a sound is not detected, then the method determines if the one or more thresholds have been exceeded in process 1215. If the head motion of the user has exceeded the one or more thresholds, then an alert is generated by the alert system in process 1217. In process 1215, if the head motion of the user did not exceed the one or more thresholds, then the alert is cleared in process 1216 by the controller. Whether the user is alerted in process 1217 or the alert is cleared in process 1216, the method returns to process 1218 to determine if the sound timeout is over. If the sound timeout is over, then the method resets the sound timeout and the one or more thresholds in process 1219 and the method begins again in process 121. If the sound timeout is not over, then the method begins again in process 121 and does not reset the sound timeout and thresholds.

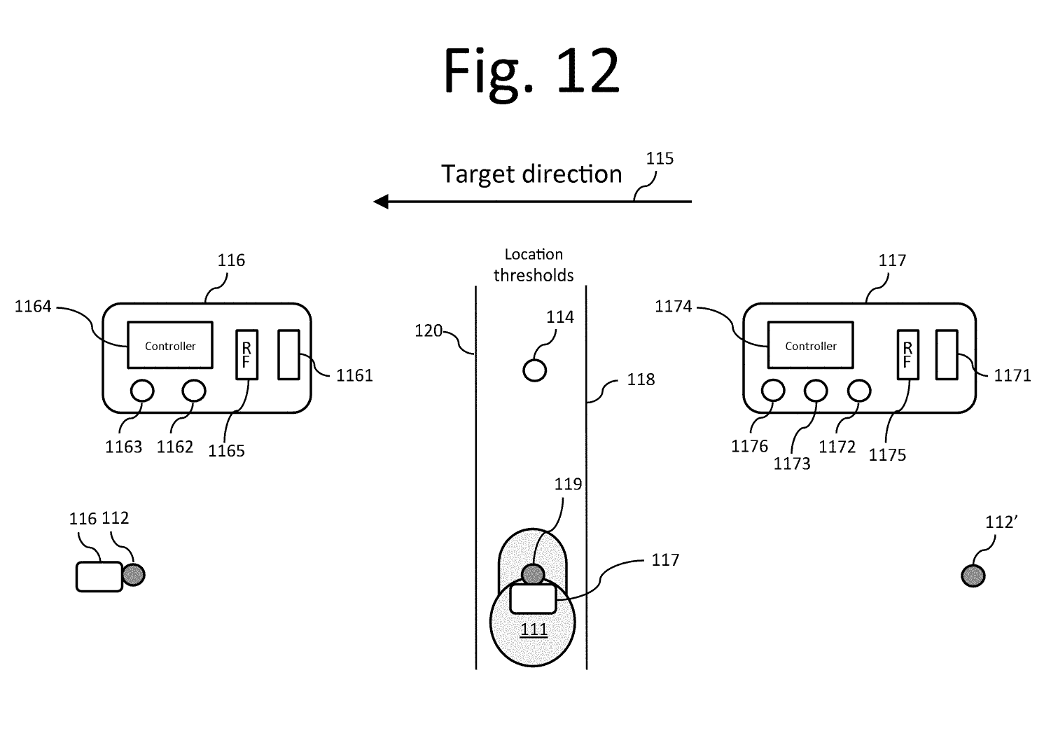

Although two or more detectors and one transmitter are discussed above, two or more transmitters and one detector, or other combinations of detectors and transmitters, may also be used. In addition, a transmitter or detector may be placed on the head of the user with a corresponding detector or transmitter placed in one or more reference locations. For example, FIG. 12 shows a head movement detection system with a control unit 117 and a detector 119 positioned on the head 111 of a user. The control unit 117 may be attached to the head 111 and a second control unit 116 may be coupled to, or in communication with, a transmitter 112. As discussed above, the control units 116, 117 may calculate the one or more thresholds 118, 120 which determine the amount the head can move over the course of a swing at a ball 114. In some embodiments, both control units 116, 117 calculate the thresholds 118, 120. As discussed above, each control unit 116, 117 may include a light 1163, 1176, (e.g., an LED), a buzzer 1162, 1173, a controller 1164, 1174, and an RF radio module 1165, 1175. Each control unit 116, 117 may be powered by a battery 1161, 1171. One of the control units 116, 117 may include a vibration element 1172. For example, the control unit 117 with a vibration element 1172 may be attached to the head 111 of the user. In this case, the vibration element 1172 may be used to alert the user if the head has moved outside the one or more thresholds 118, 120. When using one transmitter, the transmitter 112 may be placed in the forward target direction in front of the user 111 on an axis in the target direction 115. Alternatively, the transmitter 112′ may be placed in the reverse target direction behind the user 11.

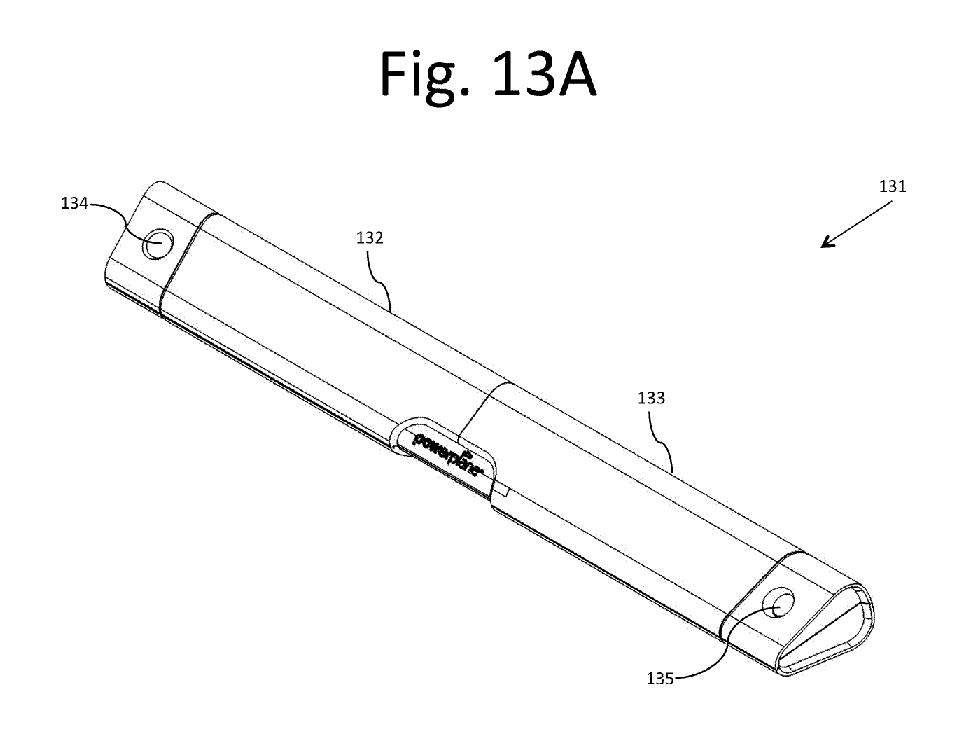

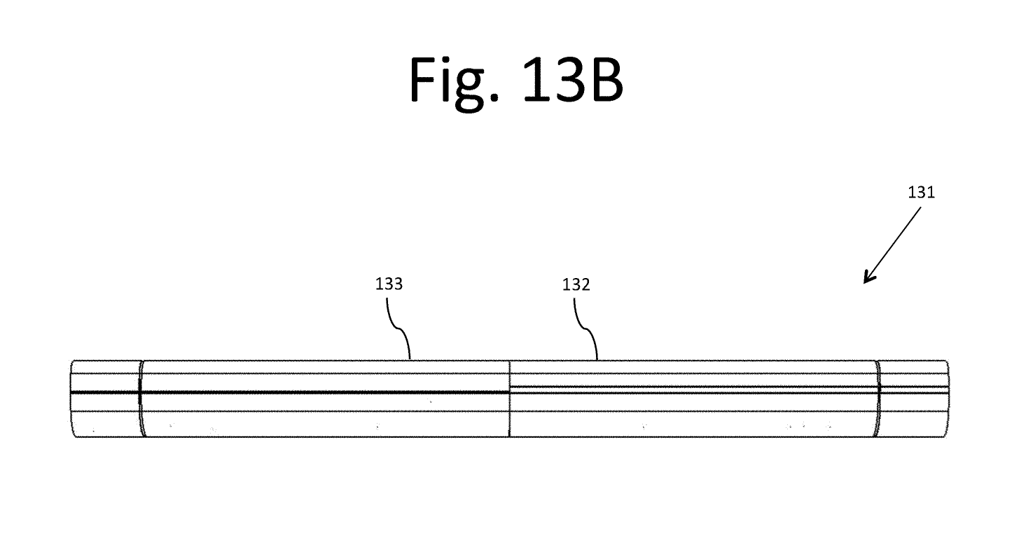

FIG. 13A schematically shows a perspective front view of a control unit 131 for an embodiment of the current invention employing the two-detector, single transmitter system described in various figures above, including 2A, 2B, 3, and 5-10. The control unit has a housing comprising two shells 132 and 133, which are assembled in slidable relation to each other. The control unit may be opened and accessed by sliding the two parts 132 and 133 apart from each other; in this view it is closed. In this embodiment, opening and closing the unit turns power on and off. The two detectors 134 and 135 are mounted in the shells 132 and 133 respectively of the control unit housing. FIG. 13B is a perspective back view of the same embodiment.

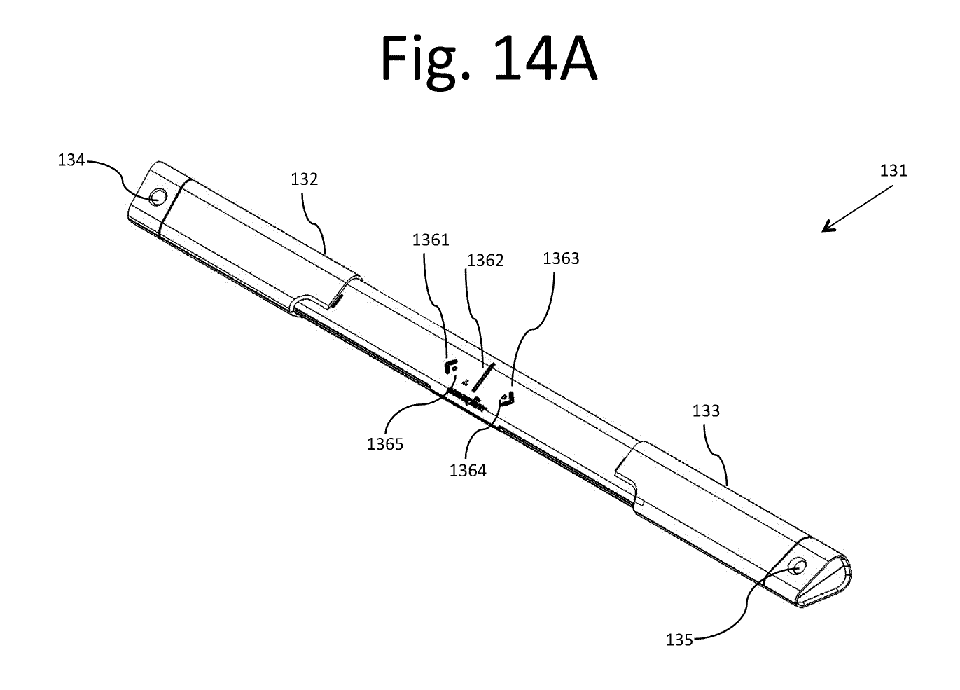

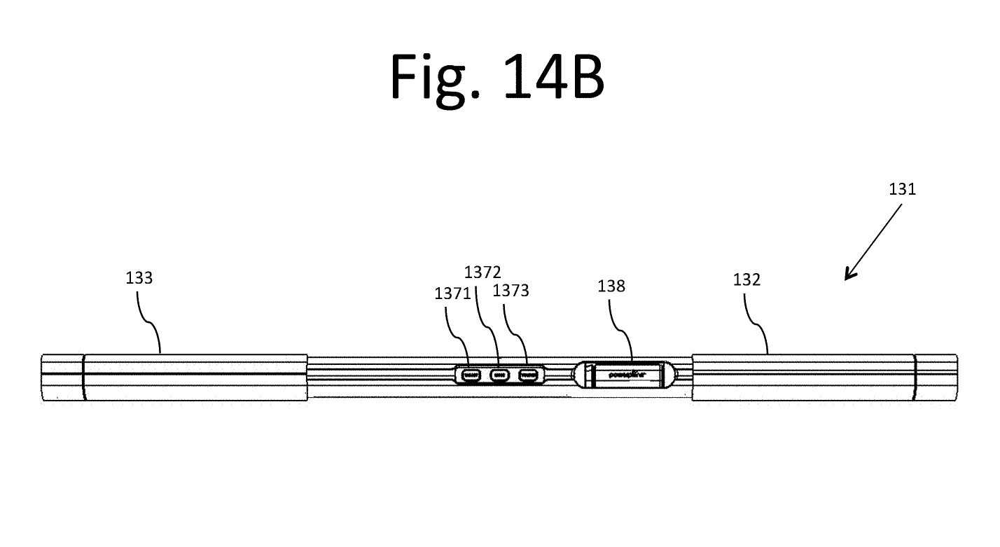

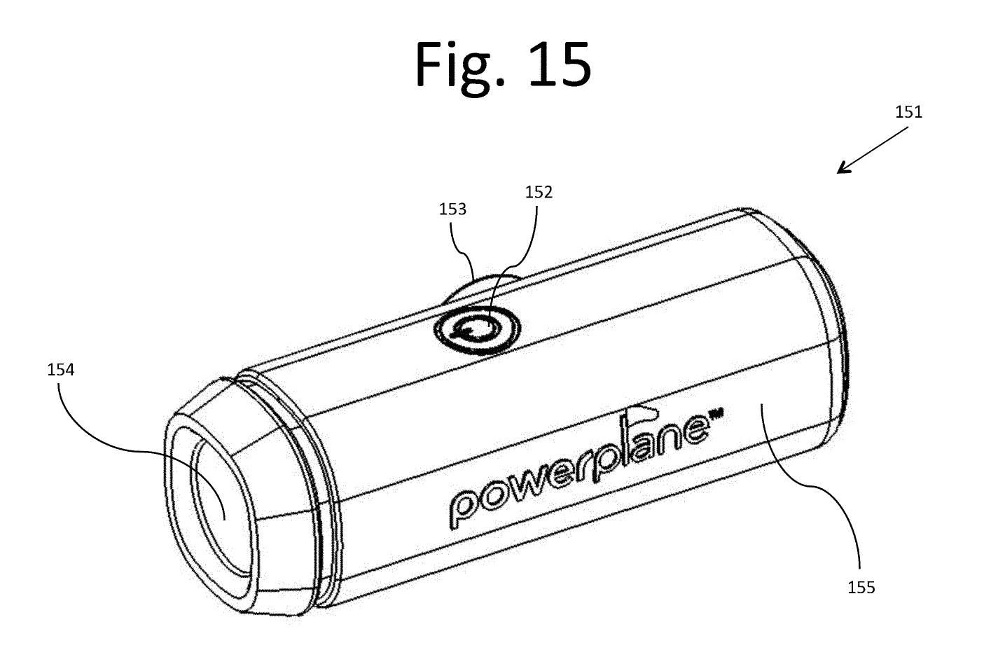

FIG. 14A schematically shows a perspective front view of a control unit 131 in the open state and reveals several other features of this embodiment. Elements 1361 through 1365 are viewports for LEDs used to communicate with the user, for example to alert the user when the user crosses location thresholds or to indicate current settings when the user configures the device. FIG. 14B is a perspective back view of the control unit 131 in the open state. Elements 1371, 1372 and 1373 are buttons the user may use to set the volume, the practice mode, and the left- or right-handed configuration of the system. The indentation 138 may be used to store the accessory unit 151 of FIG. 15 (discussed in the next paragraph) when the system is not in use. Batteries are stored inside the control unit 131 and are accessed through a port on the bottom of the control unit.

FIG. 15 is a perspective view of the accessory unit 151, for an embodiment of the current invention. The unit contains an ultrasound transmitter. Ultrasonic energy is broadcast from the unit through port 154. Power button 152 can be used to turn the unit on and off. Flange 153 holds a magnet to enable the user to easily attach the accessory to a hat clip. Batteries are stored inside the unit 151 and are accessed by sliding off the outer shell 155.

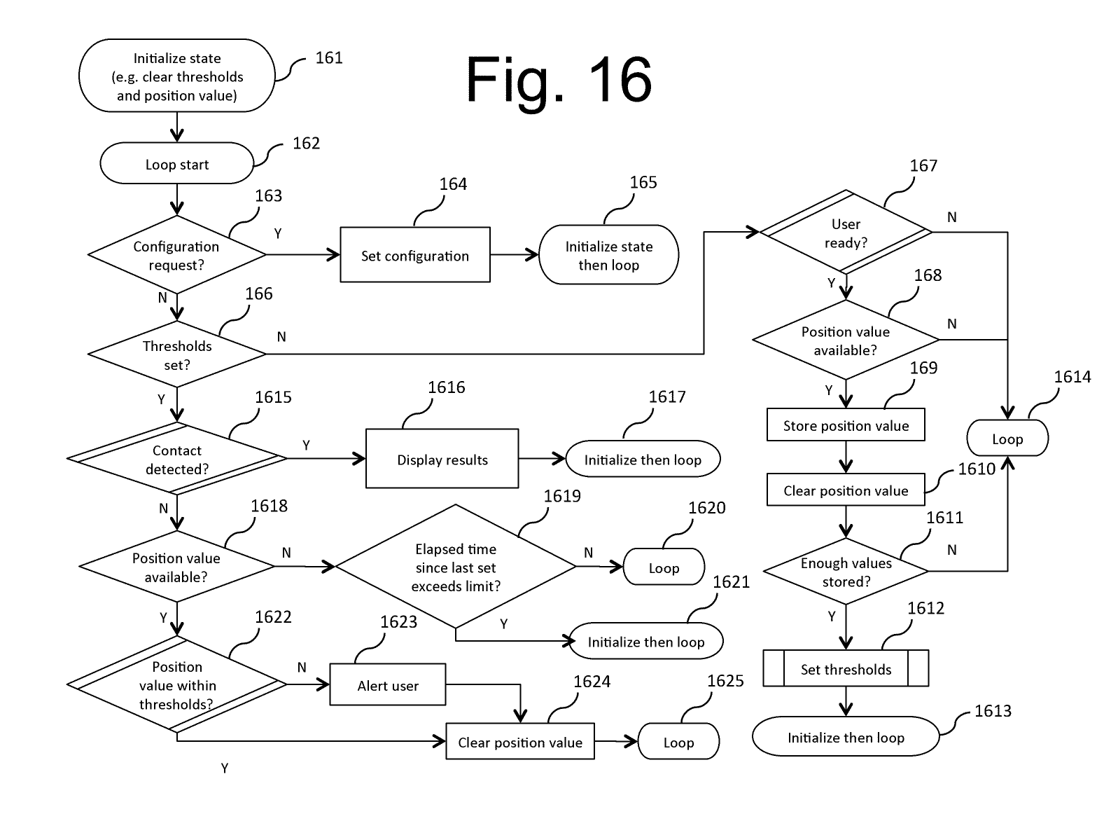

FIG. 16 is a flowchart describing an algorithm that may be used with a movement detection system, such as used with the embodiment of FIGS. 13A, 13B, 14A, 14B, and 15. It details the steps that may be performed in control units such as control unit 36 in FIG. 3, expanding on the methods of FIGS. 10 and 11 to highlight features of some embodiments of the invention. The flowchart describes the algorithm as a single procedural control loop using polling, but the steps may be implemented in software using many alternative approaches, such as an event-driven programming or interrupt-driven programming. In FIG. 16, the boxes with double-lined walls refer to flowcharts appearing in later figures.

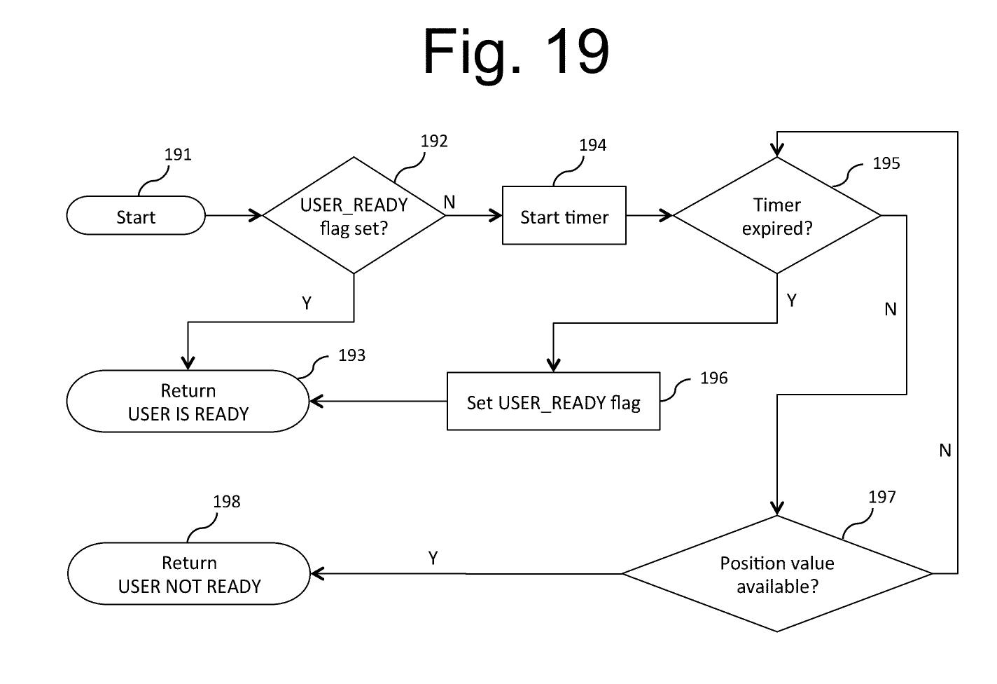

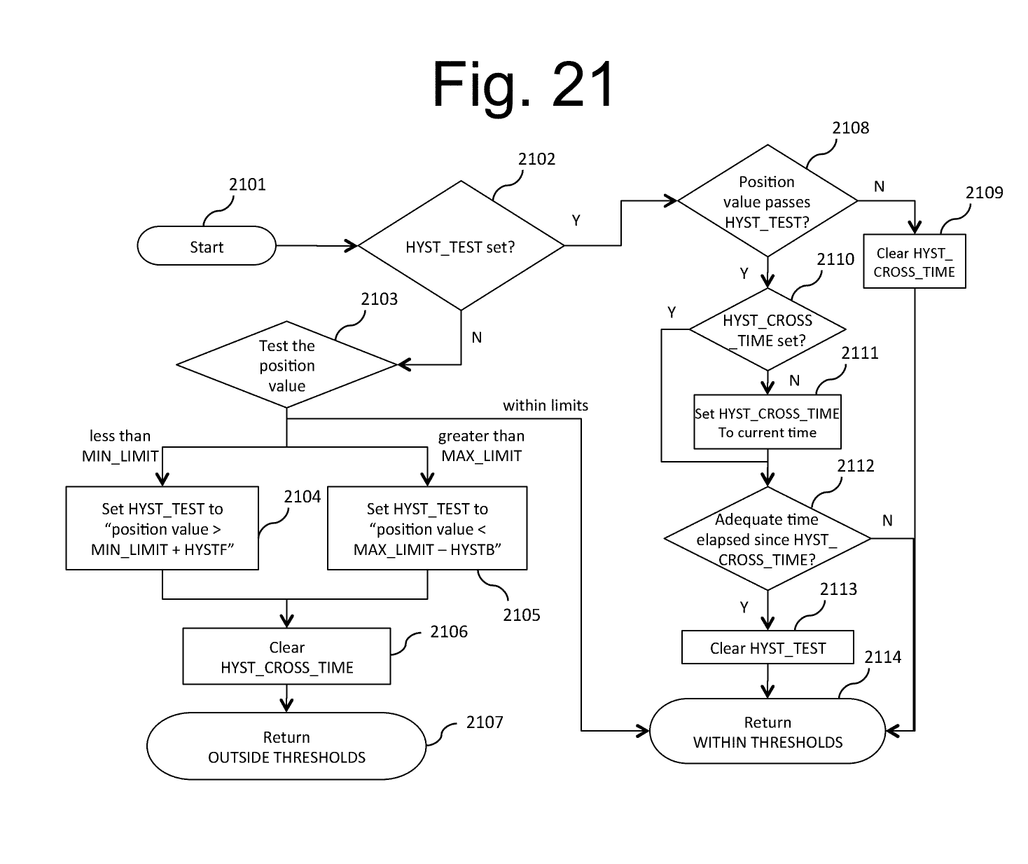

The algorithm begins at step 161 after completion of device setup, which optionally includes a device self-test. Also optionally, the control unit processor is maintained in a low-power mode until a button is pressed or a signal is received. At step 161, the variables used in this algorithm are initialized, such as clearing thresholds and the position value, and, in some embodiments, clearing the USER_READY flag, as discussed below in connection with FIG. 19, and the HYST_TEST variable discussed in connection with FIG. 21. Steps 165, 1613, 1617 and 1621 represent returning to this initialization step 161; steps 1614, 1620 and 1625 represent returning to step 162 to run through the loop again without resetting variables.

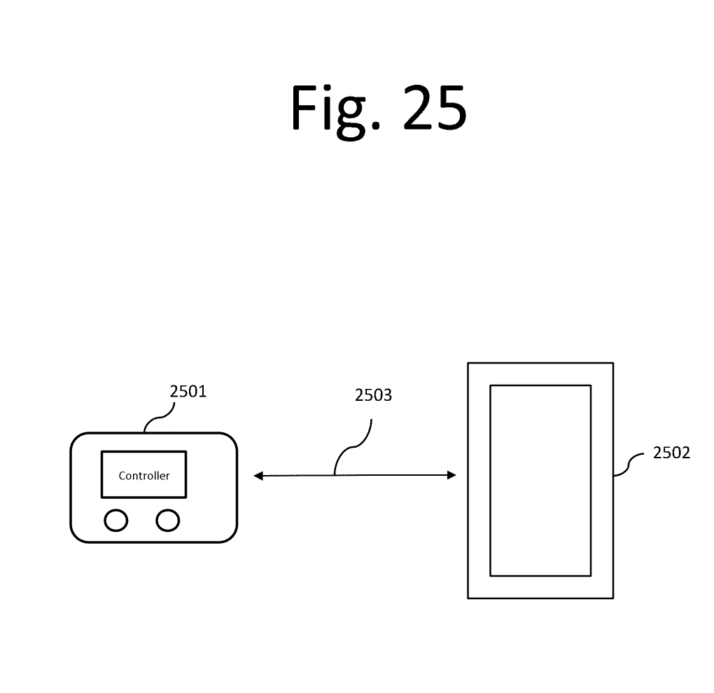

At step 163, the implementation checks if the user has requested a configuration change. In some embodiments, configurable features may include the volume of the alert system, the practice mode of the system, or whether the user is left- or right-handed. Configuration changes may be requested by depressing buttons on a controller such as 36 in FIG. 3 or interacting with an application on a computing device 2502 as shown in FIG. 25, such as a smartphone. If the user indeed requests a configuration change, then the configuration is set appropriately at step 164, and at step 165 the implementation returns to step 161 to initialize state and re-enter the loop.

At step 166, if thresholds have not been established, then at step 167 the implementation first determines if the user is ready to proceed with the user's swing. FIG. 19 details an algorithm that may be used in some embodiments to make that determination. In this algorithm, the controller maintains a USER_READY flag that is cleared at step 161 of FIG. 16. At step 192 of FIG. 19, if the USER_READY flag has already been set, processing returns to step 167 of FIG. 16 with an indication that the user is ready. Otherwise, steps 194, 195, 196, 197 and 198 of FIG. 19 have the effect of indicating that the user is ready only if a desired amount of time elapses without any position value available. In some embodiments, this may mean the user must turn his head away from the detectors for certain amount of time to indicate the user is ready to proceed. In FIG. 19, the control unit executes solely the steps of this flowchart for a certain amount of time, but the control unit may achieve the results described here by using variables and flags in the main loop or in interrupt routines.

If the user is not ready at step 167 of FIG. 16, the loop is restarted at step 162. Otherwise, at step 168 a “position value available” test occurs. The test for “position value available” at steps 168 and 1618 of FIG. 16 and step 197 of FIG. 19 may be implemented in various ways in different embodiments of the present invention. A “position value” is a quantity that can be used to detect motion in the desired direction or directions. Steps 1001, 102, 103, 104, 105 and 108 of FIG. 10 show an algorithm for an embodiment where the difference of a signal's arrival times at two detectors is used as a position value. In that case, the position value represents a hyperboloid of two sheets whose foci are the detectors. The sheets of the hyperboloid approximate flat planes in the neighborhood of the user's head. A change in the position value indicates motion from the surface of one hyperboloid to another, which can be considered to represent motion toward or away from the target. (Note that movements in a direction perpendicular to the target direction can also change this position value, but in practice only very large movements will have an appreciable effect on the position value, so this ambiguity does not adversely impact the operation of the system.) In FIG. 10, a position value becomes “available” at the beginning of step 105. It is required in FIG. 10 that each of the two detectors must receive the signal in order for the detectors to be used in calculation of the distance to the head from each detector. Because the transmitter is directional, if the user looks away from the ball, at least one of the detectors will generally lack a signal, with the result that the test for “position value available” at steps 168 and 1618 of FIG. 16 and step 197 of FIG. 19 will result in the negative. In particular, this feature allows the implementation of the algorithm illustrated by FIG. 19, wherein the user is required to look away from the ball as part of getting ready to swing and the system will not establish a starting position until after the user returns to looking at the ball. In some embodiments implementing algorithms similar to the approach illustrated in FIG. 10, the position value may only be available if the two arrival times meet other criteria, such as the magnitude of their difference being smaller than a system parameter, or their difference being similar to other recently computed differences.

Steps 121, 122, 123 and 126 of FIG. 11 show an algorithm for an embodiment where the difference in the arrival time of a sync signal and a burst signal is used as a position value. In that case, the position value represents a sphere centered on the detector, and a change in the value indicates motion from the surface of one sphere to another, which will be taken to represent motion to or away from the target. In FIG. 11, a position value becomes “available” at step 123. In some embodiments implementing algorithms similar to the approach illustrated in FIG. 11, the position value may only be available if the difference in arrival times meets other criteria, such as being smaller in magnitude than a system parameter, or being similar to other recently computed differences.

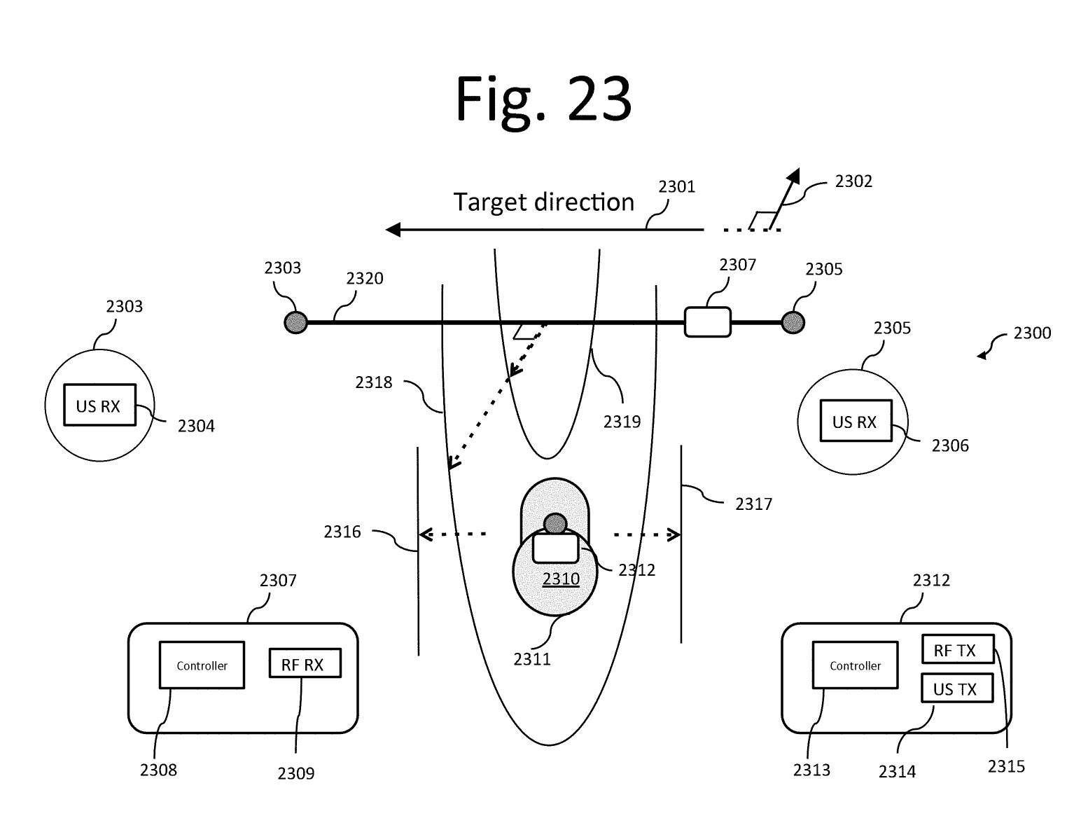

In an embodiment as shown in FIG. 23, the position value may be a pair of numbers, where one number is the difference between the arrival time of a sync signal at control unit 2307 and an ultrasound signal at detector 2303 and the second number is the difference between the arrival time of a sync signal at control unit 2307 and an ultrasound signal at detector 2305. Each difference corresponds to a sphere centered on the corresponding detector, so the pair of differences is indicative of the circle at the intersection of the two spheres when such intersection exists. That circle is centered on the line connecting the two detectors, and the plane containing that circle is perpendicular to that line, so the position value is indicative of the distance of the center of that circle from one of the detectors and the radius of that circle, and changes in the position value can measure motion parallel to (as a change in distance from one detector along the line between receivers) and perpendicular to (as a change in circle radius) the target direction. A position value becomes “available” when the control unit 2307 of FIG. 23 has detected a sync signal and detectors 2303 and 2305 of FIG. 23 have detected ultrasonic signals, all three signals detected within a specified interval of time.

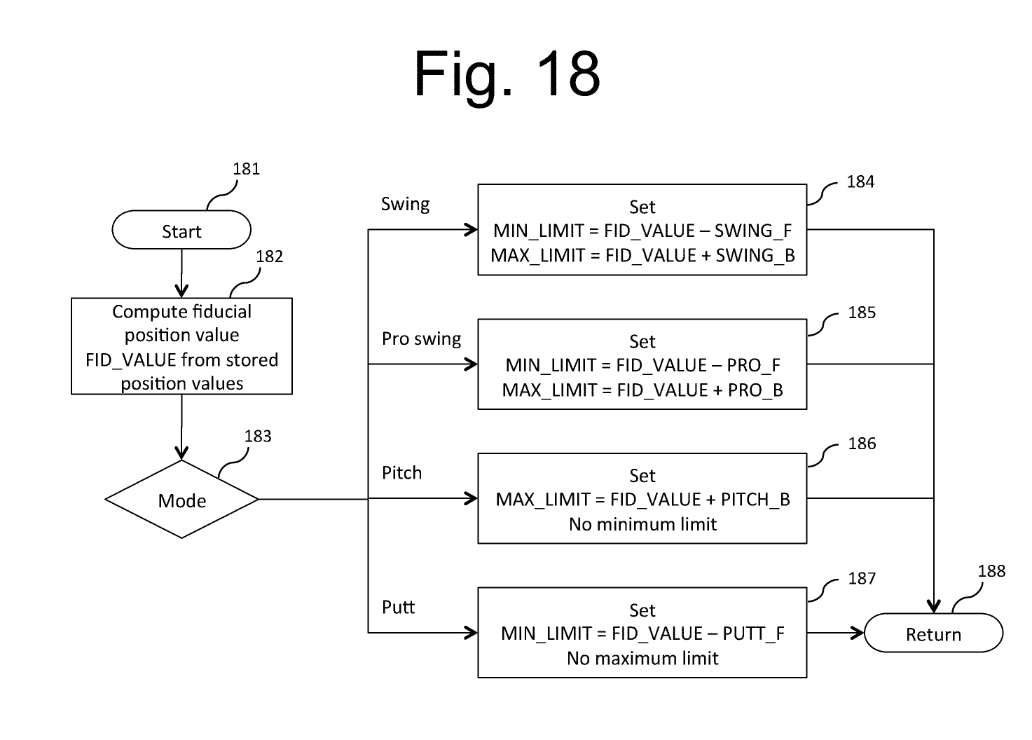

If the user is determined to be ready to swing at step 167 of FIG. 16, then repeated passes through the flowchart will execute steps 168, 169, 1610 and 1611 in order to store a set of values to use to compute, at step 1612, the location thresholds as shown in many of the figures, e.g. items 27 and 28 of FIG. 1. One algorithm for step 1612 is illustrated in FIG. 18. In step 182 of FIG. 18 a fiducial position value is computed from the stored set of position values, for example, as in step 1211 of FIG. 11, by averaging the values. Some embodiments might include an algorithm to qualify the set, and fail to compute a threshold if the set does not qualify. For example, qualification could require that the differences between stored position values are smaller than some amount, in which case the thresholds will only be set once the user has remained stationary for an adequate interval of time.

The computations shown in steps 184, 185, 186 or 1877 of FIG. 18 are for an embodiment where the position value is a number representing distance from the target, so an increase in the position value reflects motion away from the target. For example, in an embodiment of the invention using two ultrasound detectors arranged on a line parallel to the target direction where the left receiver is closer to the target, the position value may be the arrival time of a signal to the left receiver minus the arrival time of a signal to the right receiver. Embodiments allowing other configurations, such as placing the left-hand receiver closer to the target for right-handed users and placing the right-hand receiver closer to the target for left-handed users, will accommodate those configurations in this computation.

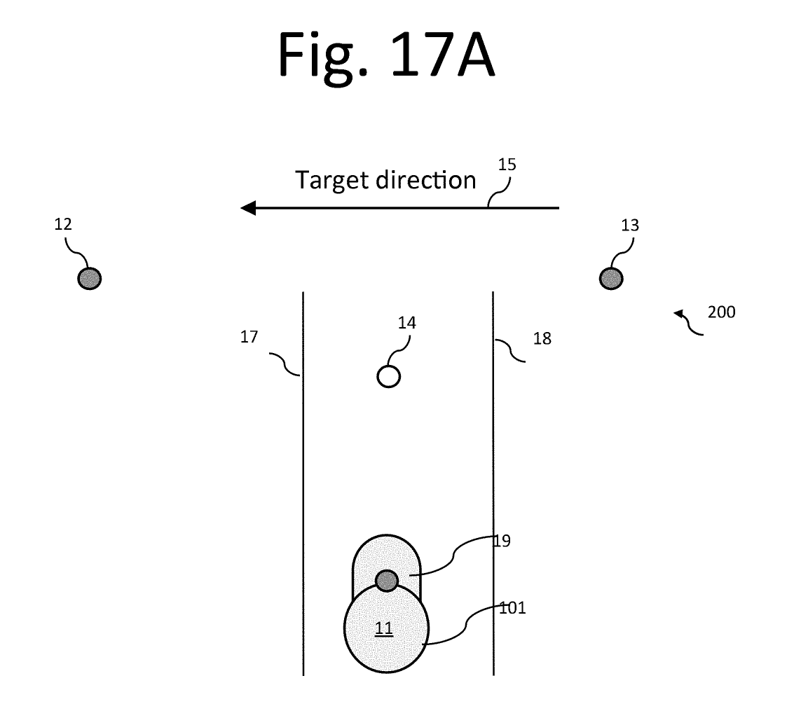

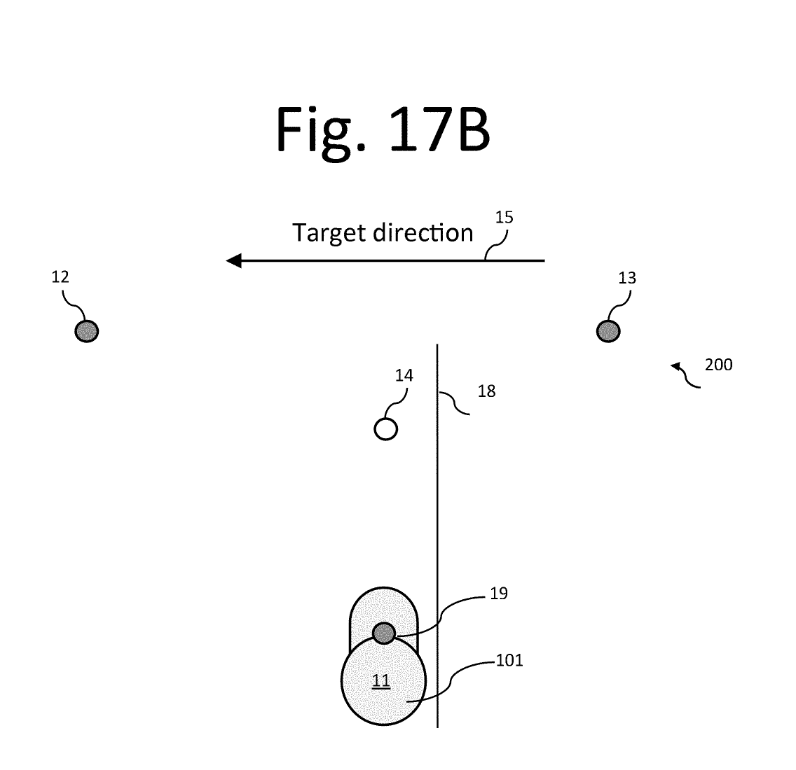

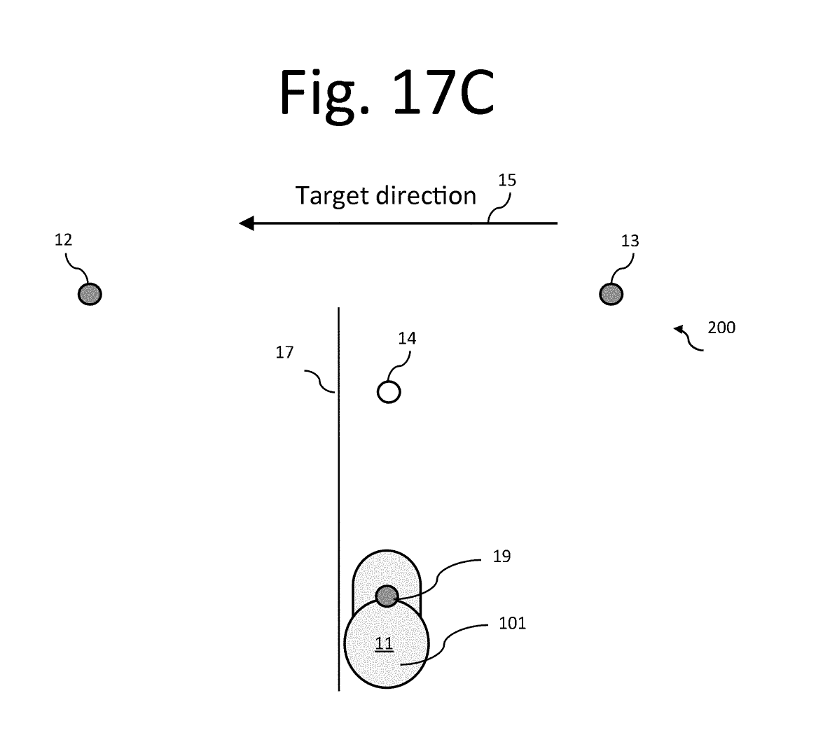

Only one of steps 184, 185, 186 and 187 is chosen, corresponding to the practice mode that the user has configured. FIG. 18 shows by way of example four practice modes: swing, pro swing, pitch and putt. If the user has configured the “Swing” practice mode at step 163 and 164 of FIG. 16, then, at step 184 of FIG. 18, minimum and maximum limits for the position value are set, corresponding to location thresholds 17 and 18 of FIG. 2A. In this case the minimum limit MIN_LIMIT variable is set to the fiducial position value minus the parameter SWING_F, and the maximum limit variable MAX_LIMIT is set to the fiducial position value plus the parameter SWING_B. SWING_F and SWING_B and the other parameters in FIG. 18—PRO_B, PRO_F, PITCH_B and PUTT_F—are determined by the designer of the embodiment and will correspond to the distance between the user's head at the start of the swing and the various location thresholds shown in FIGS. 2A, 17A, 17B and 17C. In some embodiments other factors may be considered in setting these parameters, such as the height of the user or the distance from the user to the ball. If the user configured “Pro swing” practice mode, value limits are set corresponding to FIG. 17A, where the location thresholds 17 and 18 are closer to the fiducial position than the location thresholds 17 and 18 of FIG. 2A, meeting the needs of a more advanced practitioner. If the user configured “Pitch” practice mode, then at step 186 of FIG. 18 only a maximum position value limit is set, corresponding to the single location threshold 18 of FIG. 17B, and there will be no minimum position value used, so that “pitch” practice mode can be used to alert the user only if the user moves away from the target, for example to remedy a common issue with a golf pitch swing where the golfer sways backwards during the swing. If the user selected “Putt” practice mode, then only a minimum position value limit is set and no maximum limit is set, corresponding to FIG. 17C where there is a single location threshold 17, so that the user will only be alerted if the user moves towards the target during the swing.

An embodiment as shown on in FIG. 1, with a single detector 22, may perform a computation similar to that shown in FIG. 18, to the extent it offers the same practice modes to the user as an embodiment implementing the algorithm of FIG. 18. An embodiment as shown in FIG. 23 may offer more configurable modes to the user for positioning location thresholds 2318 and 2319, which are used to alert the user to motion in a direction perpendicular to the target direction, and it may use a more complex computation than shown in FIG. 18, including the setting of position value limits corresponding to thresholds 2318 and 2319 of FIG. 23.

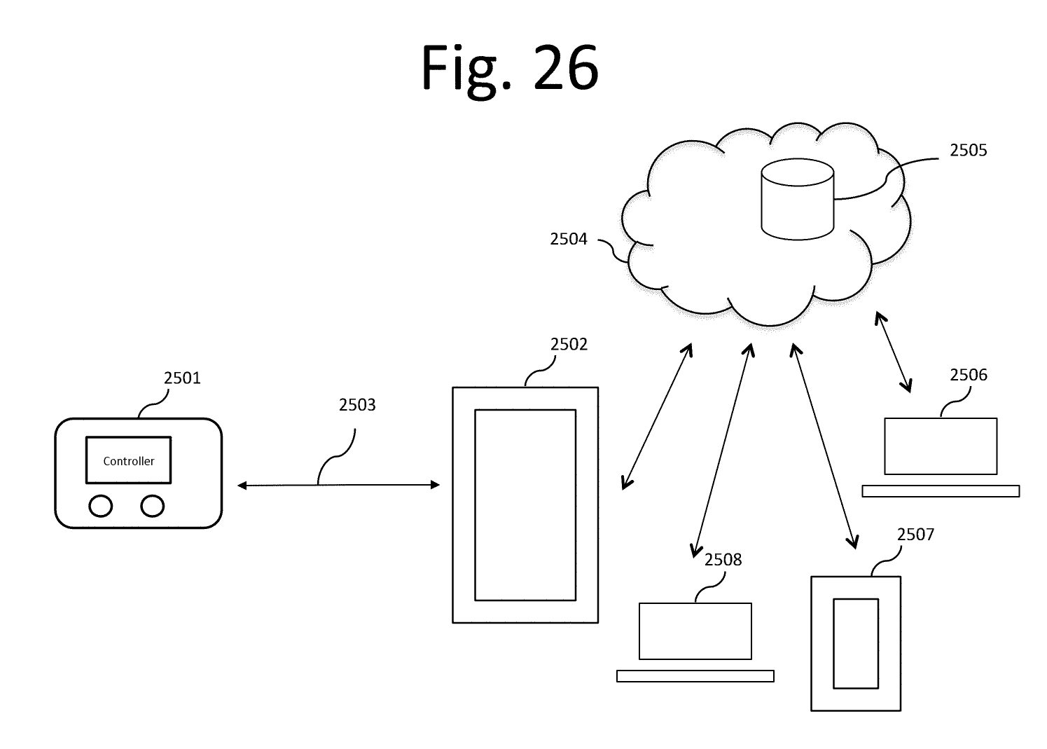

In FIG. 16, steps 1615 and beyond will only happen after thresholds have been set. If contact is detected at step 1615, then the user has finished his swing, and, at step 1616, the control unit can display results, for example illuminating LEDs indicating whether or not thresholds were crossed during the swing, and, in embodiments as per FIGS. 25 and 26, the control unit 2501 might communicate results to computing device 2502. The control unit then re-initializes state and re-enters the loop at step 162 of FIG. 16.

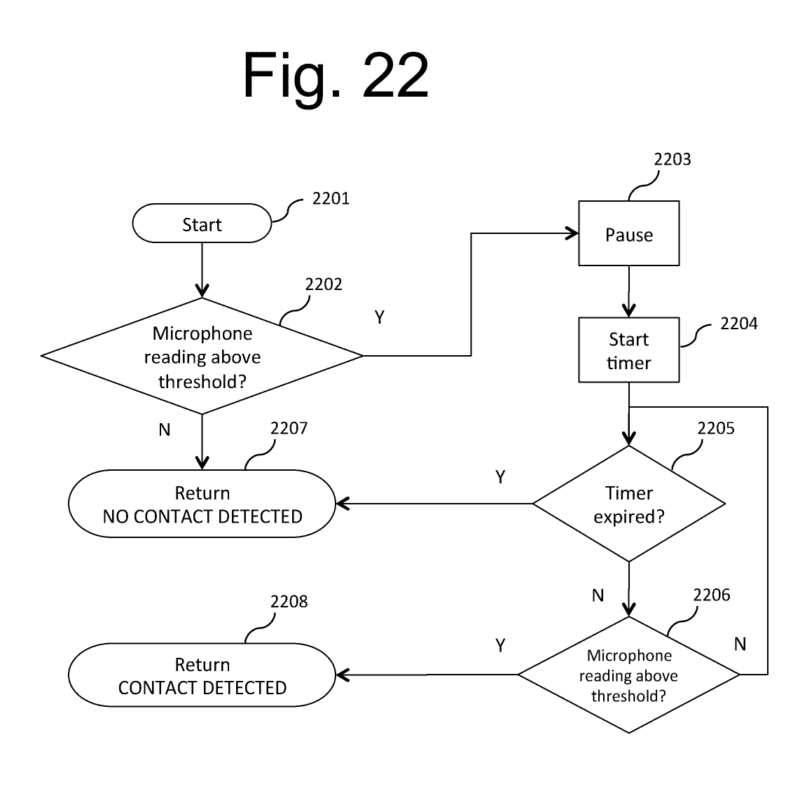

FIG. 22 shows a possible algorithm for detecting contact at FIG. 16 step 1615, by detecting sound with a microphone. To indicate that contact has been detected, the algorithm dictates that the microphone must detect sound above a threshold at FIG. 22 step 2202, then after a pause at step 2203, the microphone must detect another sound—or continuation of the same sound—within a certain time interval. The thresholds at steps 2202 and 2206 may be the same or different, and their values may depend on the characteristics of the microphone. In FIG. 22 the control unit executes solely the steps of this flowchart for a certain amount of time, but the control unit may achieve the results described here while using variables and flags in the main loop or in interrupt routines, as known to one skilled in the art.

In FIG. 16, if no contact is detected as step 1615, then if there is no position available at step 1618, the control unit, at step 1619, determines if the time elapsed since a position was last available exceeds a certain threshold; if only a short time has elapsed then the loop is re-entered, and if a long time has elapsed, state is re-initialized and then the loop is re-entered, so the user again has to indicate he is ready, followed by the control unit setting a new fiducial position value and thresholds. If there is a position available at step 1618, then at step 1622 the position value is tested to determine if the user's head is within the location thresholds set at step 1612. If the position value is outside the thresholds, then the user is alerted at step 1623 before re-entering the loop. On some embodiments the alert may be a flashing LED or buzzer, and the control unit may be programmed with techniques that allow the control unit to continue to execute the steps of the flowchart while the user is being alerted.

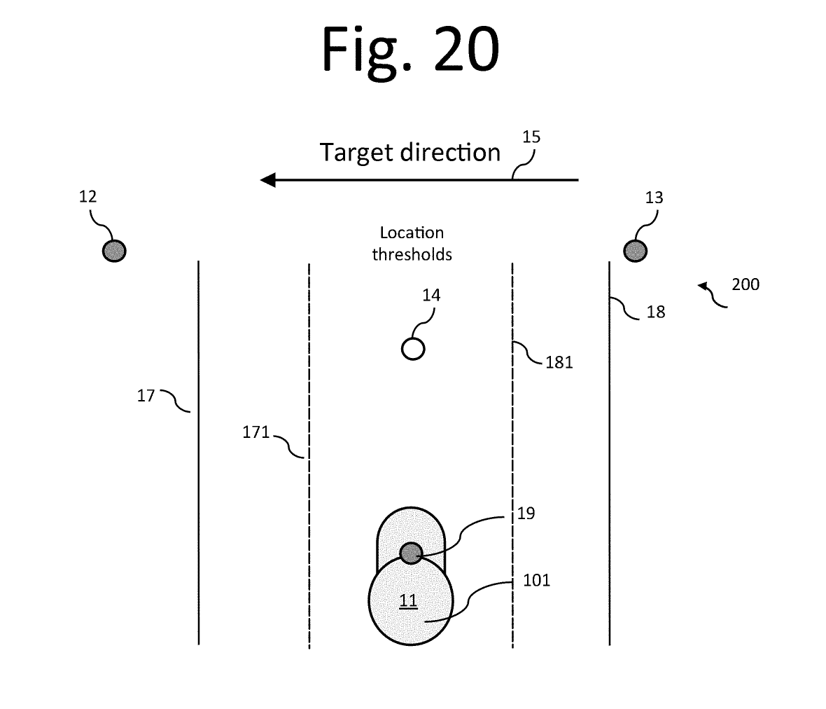

The test at step 1622 may be a simple comparison of the position value with the limits MIN_LIMIT or MAX_LIMIT or both as set at step 1612 according to the flowchart in FIG. 18. Or, the test at step 1622 may be the more complex algorithm illustrated in FIG. 20 and described in FIG. 21. The goal of this enhancement is to prevent subsequent alerts until a specified motion has occurred; the algorithm prevents the control unit from signaling repeatedly as, for example, a user's head moves quickly back and forth across a location threshold. In FIG. 20, when a user initially moves across location threshold 17 from right to left, the control unit will generate an alert. If the user then moves back to the right across 17 but not as far as 171, then again to the left across 17, an embodiment with this enhancement will not generate another alert. Or, if the user moves back across 171 but immediately moves to the left again across 17, there will be no alert if this enhancement is implemented. Only if the user moves to the right of 171 and remains to the right of 171 for at least a specified time interval will a subsequent crossing of 17 cause an alert. Similarly, after moving from left to right across location threshold 18 and generating an alert, the user must move right to left across 181 and remain to the left of 181 for a specified interval of time in order to enable the generation of alerts.

The flowchart in FIG. 21 applies to an embodiment implementing multiple practice modes as in FIG. 18. There are two variables, HYST_TEST and HYST_CROSS_TIME, used to track the state of the algorithm. HYST_TEST is used to indicate the new threshold 171 or 181 of FIG. 20 that must be crossed in order for subsequent alerts to be generated, and HYST_CROSS_TIME stores the most recent time that threshold 171 or 181 of FIG. 20 was crossed in the correct direction. When this algorithm is included in an embodiment using the approach of the flowchart in FIG. 16, HYST_TEST will be cleared whenever the device state in initialized at FIG. 16 step 161.

The current state of HYST_TEST is checked at step 2102 in FIG. 21 and, if it has not been set, at step 2103 the position value is compared to the position value limits set as per the flowchart in FIG. 18. The comparison uses the MIN_LIMIT and/or MAX_LIMIT variables. If both MIN_LIMIT and MAX_LIMIT have been computed following the flowchart in FIG. 18, then the position value is within limits if it is between them; if only MIN_LIMIT has been computed then the position value is within limits if the position value exceeds MIN_LIMIT; if only MAX_LIMIT has been computed, then the position value is within limits if the position value is less than MAX_LIMIT. If the position value is within limits, the routine finishes at step 2114, returning an indication to step 1622 of FIG. 16 that the position value is within thresholds. If the position value is outside the thresholds, then at step 2104 or 2105 HYST_TEST is set appropriately; for example if the user has crossed location threshold 17 of FIG. 20, then at step 2104 of FIG. 21 HYST_TEST will be set to test if position values are to the right of location threshold 171 of FIG. 20 by setting HYST_TEST to test that a position value is greater than MIN_LIMIT plus the parameter HYST_F. The parameters HYST_F and HYST_B of FIG. 21 are determined by the designer of the embodiment to effect the correct amount of hysteresis in the embodiment, for example increasing HYST_F means the user has to move back further to re-enable the alert. At 2107 of FIG. 21, step 1622 of FIG. 16 receives an indication that threshold 17 or 18 of FIG. 20 has been crossed and the controller can alert the user at step 1623.

If HYST_TEST is found to be set at step 2102 of FIG. 21, then at step 2108 HYST_TEST is applied to the position value. If the position value fails to meet HYST_TEST—e.g. HYST_TEST is testing for the position value to be to the right of threshold 171 of FIG. 20 and the position value is to the left of it—then at step 2109 of FIG. 20 the HSYT_CROSS_TIME variable is cleared. Because this algorithm is designed not to alert the user until certain criteria are met, at step 2114 the indication passed to step 1622 of FIG. 16 is that the position value is within thresholds, with the result that the algorithm proceeds to step 1624 of FIG. 16 and no alert is generated. Note that this indication is made at step 2114 regardless of the position value; even if the position value indicates a position to the left of 17 or the right of 18 in FIG. 20, no alert will be generated. Some embodiments may instead implement an algorithm where the user is indeed alerted that they have gone back so far as to have crossed the opposite location threshold, for example they cross threshold 17 from right to left then immediately cross threshold 18 from left to right.

If the position value passes HYST_TEST at step 2108 of FIG. 21, then if HYST_CROSS_TIME is not set, HYST_CROSS_TIME is set to the current time. The effect of the manipulations of HYST_CROSS_TIME at steps 2106, 2109, 2110 and 2111 is to keep HYST_CROSS_TIME set to the earliest time the position value agrees with HYST_TEST with no subsequent position values that fail to agree with HYST_TEST. At step 2112 the current time is compared to HYST_CROSS_TIME, and if the desired amount of time has elapsed—e.g. the user has stayed to the right of 171 of FIG. 20 for long enough—then HYST_TEST is cleared at step 2113 of FIG. 21 so that subsequent position values might again trigger alerts. The time interval used in the test at step 2112 is determined by the designer of the embodiment to effect the correct amount of hysteresis in the embodiment, for example increasing the required time interval will mean the user has to stay to the right of 171 for a longer time to re-enable the alert.

FIG. 23 shows an embodiment of the invention that can be used to detect motion with a component in direction 2301, the target direction, and motion with a component in direction 2302, perpendicular to the target direction. In this embodiment, detectors 2303 and 2305 detect ultrasonic bursts using ultrasound receivers 2304 and 2306, control unit 2307 detects electromagnetic bursts using RF receiver 2309, and transmitter 2312 generates both ultrasonic and electromagnetic bursts using ultrasound transmitter 2314 and RF transmitter 2315, controlled by controller 2313. The controller 2308 in control unit 2307 computes two numbers: the difference between the arrival time of the ultrasonic burst at detector 2303 and the arrival of the electromagnetic burst at 2307, and the difference between the arrival time of the ultrasonic burst at detector 2305 and the arrival of the electromagnetic burst at 2307. Using these two numbers, controller 2308 can then compute a position value with two components—a component indicative of a location along the line 2320 that passes between detectors 2303 and 2305 and a component indicative of a distance away from that line 2320—as described above in the discussion of “position value”. The first component, indicative of a location along the line 2320, can be used to detect if the head 2310 of the user has crossed either of the location thresholds 2316 or 2317. The second component, indicative of a distance away from the line 2320, can be used to detect if the head 2310 of the user has crossed location thresholds 2318 or 2319. Location thresholds 2318 and 2319 are circles that lie in a plane perpendicular to line 2320 and whose centers are on the line 2320. Crossing location threshold 2318, for example, might indicate that the user's head has moved up when diagnosing certain problems with the swing, or that the user's head has moved away from the ball when diagnosing other problems.

The electromagnetic burst transmitted by transmitter 2312 is used in the system 2300 to inform the control unit 2307 of the start time of the ultrasonic burst, for example by transmitter 2312 starting both the ultrasonic and electromagnetic bursts simultaneously or by starting the ultrasonic burst a fixed interval of time after the electromagnetic burst. Transmitter 2312 and control unit 2307 can use other means to share this information, such as by communicating messages over a Bluetooth or Wi-Fi connection or by both synchronizing with an external clock and transmitter 2312 sending ultrasonic bursts at specific times given by the external clock.

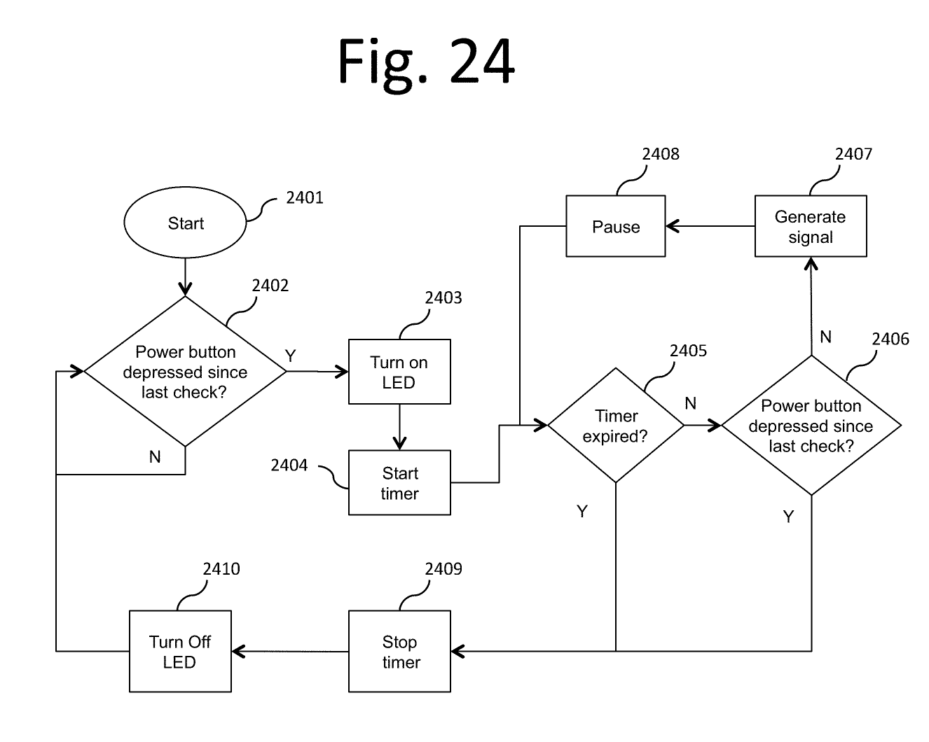

FIG. 24 is a flowchart describing an algorithm that may be used to control a transmitter in an embodiment of the invention. At step 2402, the transmitter effectively waits until a power button is depressed. In some embodiments, this may be implemented as a loop, in others it may be implemented by having a controller sleep until a button is pressed that generates an interrupt to awaken the controller. At step 2403, an LED is turned on to let the user know the device is operational; in some embodiments the LED may blink occasionally rather than stay lit, to reduce power consumption. At step 2404, a timer is started, set to the maximum interval that the device will run through iterations of the loop represented by steps 2405 through 2408. For example in some embodiments the controller will keep running through the steps of the loop for a maximum time of one hour. The controller will exit the loop represented by steps 2405 through 2408 by expiration of the timer at step 2405 or by a press on the power button detected at step 2406. If the loop is not exited, a burst signal is generated and the controller pauses for the desired interval between pulses. In some embodiments the burst may be a 40 KHz ultrasound signal for a 2 milliseconds and the pause between bursts may be 125 milliseconds. If the loop is exited, then at steps 2409 and 2410 the timer and LED are turned off, and in some embodiments the controller reenters the loop represented by step 2402 waiting for a button press; in others the controller might be put to sleep to be awakened by a button press.

In FIG. 25 the control unit 2501 establishes a communication channel 2503 with a computing device 2502, for example a smartphone, using a communications protocol, for example the Bluetooth or Wi-Fi networking protocols. The channel is used to communicate between the control unit and an application running on the computing device. The application can offer many features to enhance embodiments of the invention. The application might store the history of the user's results in order to track progress. It might store the moments that thresholds were crossed and contact was detected, or the entire history of head movement during a swing, to diagnose swing problems. It might provide an interface for configuration of the control unit, for example graphically setting the position of the location thresholds.

In FIG. 26 the system of FIG. 25 is extended by connecting the computing device 2502 to a computer network, for example the Internet. Results passed from control unit 2501 to computing device 2502 can further be forwarded to resources in the network 2504, including a server system 2505 that stores such data, and, for example, makes such data available to a web browser process running in a desktop computer 2506 of the user or, optionally of a community of users. Additionally, such data can be retrieved and analyzed by other devices with access to that resource, for example an instructor's smartphone 2507 or desktop computer 2508.

As discussed above, the head movement detection system 100, 200, 300 may use acoustic and/or electromagnetic radiation to determine whether the head has moved. Therefore, although the methods above describe measuring and calculating the time difference and calculating the thresholds using acoustic radiation, the methods may also measure and calculate the difference in strength of the signal, e.g., when using electromagnetic radiation. For example, in a system using RF radiation, the controller may store and compare RSSI values to determine whether the head has moved and to determine thresholds and values in a one or two detector system.

Although the description above discusses a user's carrying out a swing in golf, embodiments of the head movement detection system may be used in other sports requiring a swing, e.g., hockey, baseball, softball, tennis, badminton, ping pong, volleyball, racquetball, martial arts, cricket, bowling, or lacrosse. In addition, the movement detection system may be used to track body parts other than the head, for example, a knee or a hip, with the detector or transmitter placed on the part of the body to be measured.

The embodiments of the invention described above are intended to be merely exemplary; numerous variations and modifications will be apparent to those skilled in the art. All such variations and modifications are intended to be within the scope of the present invention as defined in any appended claims.

What is claimed is:

1. A method, for use in training for a sport in carrying out a swing at a ball in a target direction, for determining when, in the course of the swing by a user, a body part of the user has experienced motion having a component in the target direction, the method comprising:
repeatedly measuring over the course of the swing, using a form of radiation selected from the group consisting of acoustic and electromagnetic, a first quantity related to a first distance of the body part to a first reference location and a second quantity related to a second distance of the body part to a second reference location, the first quantity and the second quantity constituting a measurement set;
with respect to each measurement set, calculating a value that is a function of the first and second quantities, the value being indicative of body part position;
repeatedly evaluating successive values associated with each measurement set in relation to a fiducial value to determine whether the body part of the user has experienced the motion having the component in the target direction by an amount in excess of a threshold, the fiducial value selected from the group consisting of a default value, at least one previous value, an average of the values, and combinations thereof; and
in the event that the body part is determined to have experienced such motion, generating an alert to the user.

2. A method according to claim 1, further comprising:
establishing a reference body part position as defining an initial position of the body part at a beginning of the swing; and
using the reference body part position to determine the threshold.

3. A method according to claim 1, further comprising:
detecting contact with the ball; and
ending the generating of the alert when contact is detected.

4. A method according to claim 1, wherein the form of radiation is acoustic, and the acoustic radiation is ultrasonic radiation.

5. A body part movement detection system for use in training for a sport in carrying out a swing at a ball in a target direction, the system comprising:
a transmitter configured to be positioned on a body part of a user, the transmitter configured to transmit signals over the course of the swing, using a form of radiation selected from the group consisting of acoustic and electromagnetic;
a first detector, configured to be placed in a first reference location, configured to receive the signals from the transmitter and to produce a first output related to a first distance of the body part to the first reference location, the first output being indicative of body part position;
a controller, in communication with the first detector, configured to receive successive first outputs from the first detector and to evaluate the successive first outputs in relation to a fiducial value to determine whether the body part has experienced motion having a component in the target direction by an amount in excess of a threshold, the fiducial value selected from the group consisting of a default value, at least one previous value, an average of the values, and combinations thereof; and
an alert system configured to generate an alert to the user when the body part has experienced such motion.

6. A body part movement detection system according to claim 5, further comprising:
a sound detection system configured to detect contact with the ball, wherein the alert system is deactivated when contact is detected.

7. A body part movement detection system according to claim 5, wherein the form of the radiation is acoustic and the acoustic radiation is ultrasonic radiation.

8. A body part movement detection system for use in training for a sport in carrying out a swing at a ball in a target direction, the system comprising:
a transmitter configured to be positioned on a body part of a user, the transmitter configured to transmit signals over the course of the swing, using a form of radiation selected from the group consisting of acoustic and electromagnetic;
a first detector, configured to be placed in a first reference location, configured to receive the signals from the transmitter and to produce a first output, repeatedly over time, related to a first distance of the body part to the first reference location;
a second detector, configured to be placed in a second reference location, configured to receive the signals from the transmitter and to produce a second output, repeatedly over time, related to a second distance of the body part to the second reference location,
the first and second outputs at a given time constituting a measurement set;
a controller, in communication with the first and second detectors, configured to receive the measurement set at the given time and to calculate a value that is a function of the first and second outputs, the value being indicative of body part position at the given time, and configured to evaluate successive values associated with successive measurement sets in relation to a fiducial value to determine whether the body part of the user has experienced motion having a component in the target direction by an amount in excess of a threshold, the fiducial value being selected from the group consisting of a default value, at least one previous value, an average of the values, and combinations thereof; and
an alert system configured to generate an alert to the user when the body part has experienced such motion.

9. A body part movement detection system according to claim 8, further comprising:
a sound detection system configured to detect contact with the ball, wherein the alert system is deactivated when contact is detected.

10. A body part movement detection system according to claim 8, wherein the form of radiation is acoustic, and the acoustic radiation is ultrasonic radiation.

11. A body part movement detection system according to claim 8, wherein the controller is configured to establish the fiducial value corresponding to an initial position of the body part at a beginning of the swing in an adaptive manner depending on actual measurement of position of the body part at the beginning of the swing.

12. A body part movement detection system according to claim 11, wherein the fiducial value is established as an average or other combination of body part position measurements at the beginning of the swing.

13. A body part movement detection system according to claim 8, wherein the controller is configured to utilize a threshold that is manually specified.

14. A body part movement detection system according to claim 10, wherein the transmitter is an ultrasonic transmitter, the system further comprising:
an RF transmitter configured to be positioned on the body part of a user; and
an RF receiver, coupled to the controller, wherein the controller is configured to receive signals from the RF transmitter that allow the controller and the ultrasonic transmitter to synchronize.

15. A body part movement detection system according to claim 8, further comprising:
a housing in which the controller is disposed, the housing having an assembly that supports the first and second detectors, the assembly having a collapsed position in which the first and second detectors are spaced apart by a first distance and an open position in which (i) the system is operative and (ii) the first and second detectors are spaced apart by a second distance that is greater than the first distance.

16. A body part movement detection system according to claim 14, further comprising:
a housing in which the controller is disposed, the housing having an assembly that supports the first and second detectors, the assembly having a collapsed position in which the first and second detectors are spaced apart by a first distance and an open position in which (i) the system is operative and (ii) the first and second detectors are spaced apart by a second distance that is greater than the first distance.

17. A body part movement detection system according to claim 15, further comprising a switch configured, when the assembly is moved between the collapsed position and the open position, to toggle the system between an off mode and an on mode.

18. A body part movement detection system according to claim 11, wherein the transmitter is an ultrasonic transmitter and the ultrasonic transmitter is directional and the controller is further configured to defer establishing the fiducial value until after signals from any detector are absent for a specified time interval, wherein looking away from the ball has the effect of preventing an ultrasonic signal from the ultrasonic transmitter from reaching one of the detectors, so that the user must look away from the ball and then return to looking at the ball before the system will establish the fiducial value and begin monitoring the swing.

19. A body part movement detection system according to claim 8, wherein the controller is configured to provide hysteresis in resetting of the alert, so that when the alert system has generated the alert as a result of body part movement beyond the threshold, the alert system is configured not to generate a subsequent alert unless the body part has moved inside a narrower threshold and remained there for a predetermined amount of time.

20. A body part movement detection system according to claim 8, wherein the body part is a head and the transmitter is configured to be positioned on the head of the user.
